(19) United States
(12) Patent Application Publication
(10) Pub. No.: US 2012/0294837 A1
(43) Pub. Date: Nov. 22, 2012

(54) METHODS OF ISOLATING AND CULTURING MESENCHYMAL STEM CELLS

(75) Inventor: Matthew J. Hilton, Pittsford, NY (US)

(73) Assignee: UNIVERSITY OF ROCHESTER, Rochester, NY (US)

(21) Appl. No.: 13/576,745

(22) PCT Filed: Feb. 1, 2011
(86) PCT No.: PCT/US11/23369

§ 371 Date: Aug. 2, 2012

Related U.S. Application Data
(60) Provisional application No. 61/300,625, filed on Feb. 2, 2010.

(51) Int. Cl. G01N 33/566; G01N 30/00; A61P 19/10; A61K 35/12; A61P 19/04; A61P 19/08; G01N 21/64; C12N 5/0775

(52) U.S. Cl. 424/93.7; 435/7.21; 435/377; 435/325

(57) ABSTRACT

Provided herein is a relatively pure population of mesenchymal stem cells (MSCs) expressing the Notch 2 receptor (Notch 2+ a MSCs). Also provided is a method of isolating from a subject a population of Notch 2+ MSCs and a method of culturing the population of Notch 2+ MSCs. Also provided is a method of treating a subject with a disorder associated with a deficiency or defect in cells of mesenchymal lineage comprising administering a population of Notch 2+ MSCs to the subject.

This application claims the benefit of U.S. Provisional Application No. 61/300,625, filed Feb. 2, 2010, which is hereby incorporated in its entirety by this reference.

The invention was made with government support under grant numbers AR057022-01 and AR059733-01 awarded by the National Institutes of Health. The government has certain rights in the invention.

### BACKGROUND

MSCs can be isolated from various human tissues and compartments, including bone marrow, blood, adipose tissue, synovium, and fetal tissues. Human MSCs tend to grow slowly in culture, undergo cell senescence, and lose their “stem-like” properties during growth and cell passaging. Human MSC (hMSC) populations commonly express a number of cell surface markers including CD105, CD166, CD44, Stro-1 and lack expression of hematopoietic and endothelial lineage markers including CD34, CD45, and CD31. Many of these markers have been successfully used to enrich the clonogenic progenitor cell populations from bone marrow. Only a subset of bone marrow stromal cells are clonogenic and multipotent, and can therefore be identified as true MSCs. Clonogenic and multipotent MSCs have been classically identified using colony forming unit-fibroblast (CFU-F) assays. When sorted or when total bone marrow stromal cells are plated in low density, single cell-expanded colonies form. The frequency of colony forming units (CFU-Fs) is directly correlated with the incidence of clonogenic and multipotent MSCs isolated from bone marrow stromal cell populations.

### SUMMARY

Provided herein is a method of isolating from a subject a population of mesenchymal stem cells (MSCs). The method includes the steps of obtaining a biological sample comprising MSCs from the subject and selecting for MSCs expressing a Notch 2 receptor from the biological sample to obtain a population of Notch 2+ MSCs. Also provided is a relatively pure population of MSCs expressing the Notch 2 receptor (Notch 2+ MSCs).

Provided is a method of culturing a population of Notch 2+ MSCs including the step of culturing the Notch 2+ MSCs in the presence of an activator of the Notch signaling pathway. Also provided is a method of treating a subject with a disorder associated with a deficiency or defect in cells of mesenchymal lineage. The treatment method comprises administering a population of Notch 2+ MSCs to the subject.

The details of one or more embodiments are set forth in the accompanying drawings and the description below. Other features, objects, and advantages will be apparent from the description and drawings, and from the claims.

### DESCRIPTION OF DRAWINGS

FIG. 1A is a graph showing real-time (RT)-PCR gene expression levels expressed as relative gene expression of the Notch ligands, Jag1, Dll1, and Dll4 in limb-bud MSCs isolated from E11.5 mouse embryos and cultured for 6 hours, 3 days or 7 days. FIG. 1B is a graph showing RT-PCR gene expression levels expressed as relative gene expression of the Notch receptors, Notch1-3, in limb-bud MSCs isolated from E11.5 mouse embryos and cultured for 6 hours, 3 days or 7 days. FIG. 1C is a graph showing RT-PCR gene expression levels expressed as relative gene expression of the RBPjκ-dependent Notch target genes, Hes1, Hey1, and HeyL, in limb-bud MSCs isolated from E11.5 mouse embryos and cultured for 6 hours, 3 days or 7 days. Y-axis of the graphs of FIGS. 1A-1C show relative gene expression normalized to β-actin and represented in arbitrary units. hr, hours; d, days. FIGS. 1D1-1D8 are photomicrographs showing in situ hybridization gene expression analyses in limb-bud MSCs from E11.5 mouse embryos for Jag1 (FIG. 1D1), Dll1 (FIG. 1D2), Dll4 (FIG. 1D3), Notch1 (FIG. 1D4), Notch2 (FIG. 1D5), Notch3 (FIG. 1D6), Hes1 (FIG. 1D7), and Hey1 (FIG. 1D8). FIGS. 1D9 and 1D10 are photomicrographs showing in situ hybridization gene expression analyses in limb-bud MSCs from E12.0 mouse embryos for Notch2 (FIG. 1D9) and Hes1 (FIG. 1D10). Black boxes outline region of vascular canals shown in inset. Insets show high magnification of vascular canal containing blood cells and gene expression in surrounding endothelial cells for N1 and Dll4. FIG. 1E is an image of Western blot analyses for active, cleaved Notch2 protein (NICD2) isolated from limb bud-derived MSCs (LB-MSCs) cultured in the presence and absence of DAPT or from whole limb-bud (WLB) tissue.

FIGS. 2A-2C are images and graphs showing DAPT-mediated Notch inhibition enhances limb-bud MSC differentiation without biasing lineage determination. Specifically, FIGS. 2A-2C show staining and molecular analyses of limb-bud MSC cultures following continuous treatment with the Notch inhibitor, DAPT (1 μM), or vehicle. FIG. 2A shows micrographs of Alcian blue staining of limb-bud MSC micromass cartilage nodules and graphs of RT-PCR gene expression levels of the early chondrogenic markers, Sox9, Col2a1, and Agc1. FIG. 2B shows micrographs of alkaline phosphatase staining of limb-bud MSC osteogenic monolayer cultures and graphs of RT-PCR gene expression levels of the osteoblast markers, Col1a1, AP, and Oc. FIG. 2C shows micrographs of oil Red-0 staining of limb-bud MSC adipogenic monolayer cultures and a graph of RT-PCR gene expression levels of the adipocyte marker, Pparγ. Y-axis of graphs show relative gene expression normalized to β-actin and to the control. (* p<0.05 vs. control). hr, hours; d, days.

FIGS. 3A1-3A8 show images and FIG. 3B shows graphs indicating a loss of RBPjκ-dependent Notch signaling in vivo accelerates chondrogenesis during limb development. FIGS. 3A1 and 3A2 show Alcian blue staining of wild-type (WT) and Prx1Cre/Rbpjκ^{f/f} (RBPjκ) E12.5 hindlimbs. FIGS. 3A3-3A8 show in situ hybridization gene expression analyses of the chondrogenic marker genes Sox9 (FIGS. 3A3 and 3A4), Col2a1 (FIGS. 3A5 and 3A6), and Agc1 (FIGS. 3A7 and 3A8). FIG. 3B shows graphs of RT-PCR gene expression levels from whole limb-buds of WT and RBPjκ mutant E12.5 hindlimbs. Y-axis of graphs show relative gene expression normalized to β-actin and to the WT control. (* p<0.05 vs. control).

FIGS. 4A1-4A6 and 4B1-4B10 show images and FIG. 4C shows graphs indicating sustained activation of Notch signaling suppresses MSC differentiation during skeletal development. FIGS. 4A1-4A6 show Alcian blue/Alizarin red staining of wild-type (WT) and Prx1Cre/Rosa-NICD^{F/+} (NICD) mutant E18.5 whole skeletons (FIGS. 4A1 and 4A2), forelimbs (FIGS. 4A3 and 4A4), and hindlimbs (FIGS. 4A5 and 4A6). Black arrows indicate NICD mutant forelimb and hindlimb. FIGS. 4B1 and 4B2 show Alcian blue staining of WT and NICD hindlimbs at E12.5. FIGS. 4B3-4B8 show in situ hybridization gene expression levels of the chondrogenic marker genes Sox9 (FIGS. 4B3 and 4B4), Col2a1 (FIGS. 4B5 and 4B6), and Agc1 (FIGS. 4B7 and 4B8). FIGS. 4B9 and 4B10 show Gfp expression monitored to assess NICD expression and activity in WT (FIG. 4B9) and NICD mutant (FIG. 4B10) hindlimbs. FIG. 4C shows graphs of RT-PCR gene expression levels from whole limb-buds for the chondrogenic markers, Sox9, Col2a1, Agc1, and Runx2 and the RBPJκ-dependent Notch target genes, Hes1, Hey1, and HeyL. Y-axis of graphs show relative gene expression normalized to β-actin and to the WT control. (* p<0.05 vs. control). d, digits; r, radius; u, ulna; h, humerus; s, scapula; t, tibia; fi, fibula; fe, femur; il, illium; pu, pubic.

FIGS. 5A1-5A6 and 5C1-5C2 show images and FIGS. 5B and 5C3-5C4 show graphs showing sustained activation of Notch signaling in the limb mesenchyme does not significantly affect limb patterning or apoptosis, but increases MSC proliferation during limb development. FIGS. 5A1-5A6 show in situ hybridization analyses of wild-type (WT) (FIGS. 5A1, 5A3 and 5A5) and Prx1Cre/Rosa-NICD^{f/+} mutant (NICD) (FIGS. 5A2, 5A4, and 5A6) limb-bud sections at E11.0. Gene expression patterns were analyzed for the limb-bud outgrowth and patterning markers: Fgf8 (FIGS. 5A1 and 5A2), Fgf10 (FIGS. 5A3 and 5A4), and Ptc1 (FIGS. 5A5 and 5A6). FIG. 5B shows fluorescent TUNEL staining and statistical analyses of MSC apoptosis performed on WT and NICD mutant sections at E11.0. BrdU immunohistochemistry (FIGS. 5C1 and 5C2) and statistical analyses of MSC proliferation (FIG. 5C3) were performed on WT (FIG. 5C1) and NICD mutant (FIG. 5C2) sections at E11.5. (* p<0.05 vs. control). AZ, apical zone. Dashed boxes denote regions analyzed for MSC proliferation. FIG. 5C4 shows RT-PCR levels of cyclinD1 using RNA derived from NICD mutant and control limb-buds at E11.5.

FIGS. 6A1-6A4 and 6B1-6B15 show images indicating Notch signaling suppresses MSC differentiation in an RBPJκ-dependent manner. FIGS. 6A1-6A4 show Alcian blue/Alizarin red staining of wild-type (WT); Prx1Cre/Rosa-NICD^{f/+} (NICD); Prx1Cre/Rbpjκ^{f/f} (RBPjκ); and Prx1Cre^{f}/Rosa-NICD^{f/+}/Rbpjκ^{f/f} (NICD; RBPjκ) mutant E18.5 whole skeletons. Black arrows indicate NICD mutant forelimb and hindlimb. Gray arrows mark the length of WT, RBPjκ, and NICD; RBPjκ tibiae. Asterisks identify location of the parietal bones. FIGS. 6B1-6B3 show Alcian blue staining of WT, NICD, and NICD; RBPjκ littermate hindlimb sections at E12.5 (B1-B3). FIGS. 6B4-6B12 show in situ hybridization gene expression analyses of the chondrogenic marker genes Sox9 (FIGS. 6B4-6B6), Col2a1 (FIGS. 6B7-6B9), and Agc1 (FIGS. 6B10-6B12). FIGS. 6B13-6B15 show Gfp expression monitored to assess NICD expression and activity in WT (FIG. 6B13), NICD mutant (FIG. 6B14), and NICD; RBPjκ rescue (FIG. 6B15) hindlimb sections.

FIGS. 7A1-7A6 show images and FIG. 7B shows graphs indicating Hes1 is a critical RBPjκ-dependent Notch target gene regulating MSC differentiation and chondrogenesis. FIGS. 7A1-7A6 show Alcian blue staining of control infected (FIGS. 7A1, 7A3, and 7A5,) and Hes1 shRNA infected (shHes1) (FIGS. 7A2, 7A4, and 7A6) limb-bud MSC cells cultured in micromass for 3, 5, or 7-days. FIG. 7B shows RT-PCR gene expression levels for the chondrogenic markers Sox9, Col2a1, Agc1 during in vitro chondrogenesis following knock-down of Hes1. Y-axis of graphs show relative gene expression normalized to β-actin and to the control at day 3. (* p<0.05 vs. control). d, days.

FIG. 8 is a graph showing apoptotic cell counts in E11.5 sections from WT and NICD mutant limb mesenchyme. Using activated caspace-3 immunohistochemistry, the data show sustained activation of Notch signaling in the limb mesenchyme does not affect MSC apoptosis.

FIGS. 9A1-9A6 and 9B1-9B6 show images and FIGS. 9C and 9D show graphs indicating Hes1 is a critical regulator of MSC differentiation in a C3H10T1/2 model of chondrogenesis. FIGS. 9A1-9A6 and 9B1-9B6 show Alcian blue staining of control infected (FIGS. 9A1, 9A3, and 9A5,), Hes1 shRNA infected (shHes1) (FIGS. 9A2, 9A4, and 9A6), control transfected (FIGS. 9B1, 9B3, and 9B5), and Hes1 transfected (CMV-Hes1) (FIGS. 9B2, 9B4, and 9B6) C3H10T1/2 cells cultured in micromass for 5, 10, or 14-days. FIGS. 9C and 9D show RT-PCR gene expression levels for the chondrogenic markers Sox9, Col2a1, Agc1 and the Notch target gene, Hes1 during in vitro chondrogenesis following knock-down of Hes1 (FIG. 9C) or over-expression of Hes1 (FIG. 9D). Y-axis of graphs show relative gene expression normalized to β-actin and to the control at day 5. (* p<0.05 vs. control). d, days.

FIGS. 10A and 10B are graphs showing Notch molecules expressed in hMSCs. Gene expression is normalized to beta-actin and represented in arbitrary units

FIGS. 11A-11C are graphs showing recombinant Jagged1 induction of multipotent stem cell markers and hMSC proliferation. FIG. 11A shows gene expression levels for Notch components and regulators of stem cell multipotency in hMSCs at passage 1 (P1) and passage 15 (P15). FIG. 11B shows gene expression levels for Notch target genes and regulators of stem cell multipotency in hMSCs cultured on control IgG or Jag1 coated plates. All gene expression is normalized to beta-actin and then normalized to P1 controls (FIG. 11A) or IgG controls (FIG. 11B). FIG. 11C shows BrdU ELISA assay measuring proliferation of hMSCs cultured on IgG control or Jag1 coated plates.

FIGS. 12A and B show flow cytometry data for hMSC cell surface marker, CD105 (A), and the Notch receptor, Notch2 (B), following passages 2 and 10 in standard hMSC culture conditions.

FIG. 13A-C show that Jag1-mediated Notch activation in Notch2-selected hMSCs induces stem cell regulators, cell proliferation, and stem cell expansion. FIG. 13A shows real-time RT-PCR gene expression analyses for Notch signaling molecules (Notch2 and Hes1), important stem cell regulatory molecules (Oct4, Sox2, and Nanog), and a marker of cell proliferation (CycD1) in total hMSCs and Notch2-selected hMSCs cultured on Jag1 coated plates. FIG. 13B shows a BrdU ELISA assay performed on total, Notch2-negative, and Notch2-positive hMSCs cultured on Jag1 coated plates. FIG. 13C shows a CFU-F assay performed on total, Notch2-negative, and Notch2-positive hMSCs following culture on Jag1 coated plates. FIGS. 14A-D show Notch2-selected hMSCs display enhanced chondrogenic and osteogenic properties following Jag1-mediated maintenance and expansion. FIGS. 14A and C show real-time RT-PCR gene expression analyses for chondrogenic (Sox9, Col2a1, and Agc1) (A) and osteogenic (Col1a1, Ap, and Oc) (C) marker genes from total, Notch2-negative, and Notch2-positive hMSCs after being cultured in chondrogenic or osteogenic conditions for two to three weeks. FIG. 14B shows Alcian Blue staining of total, Notch2-negative and positive hMSCs (Passage 2) following chondrogenic differentiation. FIG. 14D shows AP staining of Notch2-negative and positive hMSCs (Passage 2 and 5) following osteogenic differentiation. hMSCs were initially cultured on Jag1 coated plates for two passages (3-4 days/passage).

### DETAILED DESCRIPTION

To determine the exact role and mode of action for the Notch pathway in mesenchymal stem cells (MSCs), tissue specific loss-of-function (Prx1Cre; Rbpjκ^{f/f}), gain-of-function (Prx1Cre; Rosa-NICD^{f/+}) and genetic rescue mice (Prx1Cre; Rosa-NICD^{f/+}; Rbpjκ^{f/f}) were generated and analyzed for defects in MSC proliferation and differentiation during early limb development. The results are presented in Example 1 below. These data show that Hes1 is the primary RBPjκ-dependent Notch target gene of the Hes/Hey family expressed in MSCs and required for the Notch mediated suppression of MSC differentiation during chondrogenesis. Further, these data demonstrate that the RBPjκ-dependent Notch signaling pathway is critical for the maintenance and expansion of MSCs during skeletal development. Thus, manipulation of the Notch pathway provides a means to maintain, expand, and regulate the differentiation of MSCs for the purpose skeletal repair and tissue engineering applications that utilize MSC populations. Controlled Notch activation of hMSCs promotes the maintenance and expansion of hMSCs, while preserving their chondrogenic, osteogenic, and adipogenic differentiation potential. Accordingly, disclosed herein are relatively pure populations of MSCs and methods of isolating and culturing MSCs.

Provided is a relatively pure population of MSCs expressing the Notch 2 receptor (Notch 2+ MSCs). As used herein, the term relatively pure means that at least 90, 91, 92, 93, 94, 95, 96, 97, 98, 99 or 100% of the MSCs in the population express Notch 2. Optionally, the Notch 2+ MSCs maintain the capacity to expand through multiple passages. Optionally, the Notch 2+ MSCs express one or more additional markers associated with mesenchymal stem cells selected from the group consisting of CD105, CD106, CD156, CD44, CD29, CD166, Stro-1, FGF10, Prx1, Oct4, Sox2, and Nanog. Optionally, the Notch 2+ MSCs express CD105 and CD156. Optionally, the Notch 2+ MSCs do not express one or more markers associated with hematopoietic or endothelial cell lineage selected from the group consisting of CD34, CD45, CD14, and CD31.

The relatively pure population of Notch 2+ MSCs is stable in non-differentiating culture conditions. As used herein, non-differentiating culture conditions include, but are not limited to, culture conditions that promote proliferation without promoting differentiation. For example, the cells can be maintained in medium, e.g. DMEM, RPMI, and the like, in the presence of fetal bovine serum or serum-free replacement without differentiation.

Specifically, provided is a method of isolating from a subject MSCs. The method includes the steps of obtaining a biological sample comprising MSCs from the subject and selecting for MSCs expressing a Notch 2 receptor from the biological sample to obtain a population of Notch 2+ MSCs. Also provided is a relatively pure population of Notch 2+ MSCs made by the provided methods. The MSCs maintain the capacity to expand through multiple passages. The MCSs can be passaged at least about 5, 10, 15 or 20 times or any number of times between 5 to 20. Optionally, the MSCs can be passaged 10 or more times. For example, the MSCs can be passaged 10, 11, 12, 13, 14, 15, 16, 17, 18, 19 or 20 times.

As used herein, the terms passaged or passaging refers to the process of sub-culturing cells. The methods and materials for culturing and passaging cells are known. For example, cells are grown on a substrate, e.g., in a dish or plate, with media in an incubator. During passaging, the growth media is removed, and the cells may be washed, followed by the addition of an agent to detach the cells from the substrate. The detached cells are suspended and an appropriate number of cells in suspension is then transferred to new substrates, fresh medium is added, the new substrates are put in the incubator, and the cycle begins again. Cells are often kept less than 100% (log phase of growth) but more than 10% confluent. Cells may die if they are too few or much too crowded.

The selection step is carried out using any one of a variety of methods including, but not limited to, flow cytometry, magnetic bead separation, panning, fluorescence activated cell sorting (FACS) or affinity chromatography. For example, flow cytometry, or FACS, can be used to separate cell populations based on the intensity of fluorescence, as well as other parameters such as cell size and light scatter.

The selection step is, optionally, carried out using a Notch 2 receptor antibody or other Notch 2 receptor ligand. Optionally, the antibody or ligand is bound to a substrate, which can be, for example, a mobile or immobile solid support. Optionally, the mobile solid support is a fluorescent bead. Optionally, the immobile solid support is a column or a plate. The sample is contacted with the substrate and, either the substrate with the Notch 2+ cells is sorted from substrate lacking the Notch 2+ cells, or the bound MSCs in the sample are isolated from the substrate, e.g. with a competitive binding step. Fluorescent labels or other labeling means can be used to sort the MSCs. With sorting techniques like FACS, the various populations of MSCs can be sorted to have the specifically desired expression profiles.

The sample from the subject is selected from an MSC-containing sample, e.g., from the group consisting of bone marrow, adipose tissue, synovium, periosteum, perichondrium, cartilage, dental tissue, placental tissue, liver tissue, muscle tissue, lung tissue, heart tissue, connective tissue, and spleen tissue.

The isolated Notch 2+ MSCs are collected, for example, in any appropriate medium that maintains the viability of the cells. Optionally, the medium is located in a collection vessel, such as a tube. Various media are commercially available and may be used, including, but not limited to, Dulbecco's Modified Eagle Medium (DMEM), Hanks' Buffered Salt Solution (HBSS), Dulbecco's Phosphate Buffered Saline (dPBS), Roswell Park Memorial Institute (RPMI) medium, Iscove's medium, and the like, optionally, supplemented with fetal calf serum.

Also provided is a method of culturing the population of Notch 2+ MSCs including the step of culturing the MSCs in the presence of an activator of the Notch signaling pathway. Optionally, the culture conditions are such that the population of Notch 2+ MSCs is expanded. Various media are commercially available and may be used to culture MSCs, including, but not limited to, DMEM, HBSS, dPBS, RPMI medium, Iscove's medium, and the like, optionally, supplemented with fetal calf serum.

Optionally, the activator of the Notch signaling pathway is selected from the group consisting of delta-like 1, delta-like 3, delta-like 4, Jagged1, Jagged 2, Dlk1/Pref1, DNER, Contactin1 (F3), Contactin6 (NB3), CCN3/NOV, MAGP1, and MAGP2. Optionally, the activator of the Notch signaling pathway is an intracellular domain of a Notch receptor. Optionally, the Notch receptor is Notch 1, Notch 2, Notch 3, or Notch 4. The activator of the Notch signaling pathway can be partially or completely immobilized on a culture dish. Alternatively, the activator can be soluble in the culture medium.

Notch activation can be induced by a ligand, which causes cleavage and release of the Notch intracellular domain (ICD). The NICD translocates to the nucleus, interacts with RBPjk, and activates target genes. Notch signaling in MSCs can also be activated by directly expressing a Notch ICD. Notch ICD expression can be provided using any means for expressing a peptide in a cell, for example, using an expression vector (e.g., a viral vector). Expression of the Notch ICD can be transient or stable.

The culturing method can also include the step of culturing the population of Notch 2+ MSCs in the presence of one or more differentiating agents. Notch activation is “turned off” to allow the cell to differentiate. Optionally, the one or more differentiating agents selectively induce differentiation into chondrogenic, osteogenic or adipogenic lineages. Culturing the Notch 2+ MSCs under differentiating culture conditions is carried out by culturing or differentiating MSC in a growth environment that enriches for selected cells with the desired phenotype, e.g. osteoblasts, adipocytes, chondrocytes, or the like. Thus, the culture medium may include agents that enhance differentiation to a specific lineage. For example, osteogenic differentiation may be enhanced by culturing MSCs in medium comprising 8-glycerol phosphate, ascorbic acid and retinoic acid (Cowan et al. (2005) Tissue Engineering 11:645-658). Adipogenic differentiation may be enhanced, for example, by culturing the MSCs in a medium comprising dexamethasone, indomethacin, 3-isobutyl-1-methylxanthine (IBMX), and insulin, then maintaining in growth media with insulin. Myocyte differentiation may be enhanced, for example, by culturing in a medium comprising 5-azacytidine (Fukuda et al. (2001) Artificial Organs 25:187), or in a medium comprising horse serum, dexamethasone, and hydrocortisone (Eun et al. (2004) Stem Cells 22:617-624). Chondrocyte differentiation may be enhanced, for example, by culturing in a medium comprising dexamethasone, ascorbic acid 2-phosphate, insulin, transferrin, and selenous acid, with or without TGF-θ₁ (Williams et al. (2003) Tissue Engineering 9(4):679). Following differentiation in culture, the cells obtained may be used directly, or may be further isolated, e.g. in a negative selection to remove MSCs and other undifferentiated cells. Further, enrichment for the desired cell type may be obtained by selection for markers characteristic of the cells, e.g. by flow cytometry, magnetic bead separation, panning, and the like, as is known.

Provided is a method of treating a subject with a disorder associated with a deficiency or defect in cells of mesenchymal lineage comprising administering a population of Notch 2+ MSCs to the subject. The population of Notch2+ MSCs are derived from the same or a different subject.

The Notch 2+ MSCs are administered to the subject as appropriate. For example, the Notch 2+ MSCs are injected into the subject at or near the site of the bone or cartilage defect or administered to the subject systemically. The Notch 2+ MSCs are administered in a manner that permits them to graft or migrate to the intended tissue site and reconstitute or regenerate the functionally deficient area. Optionally, targeting molecules on the surface of the MSCs are used to promote proper migration to the desired site. MSCs are used, for example, for engineering cartilage, growth plate, bone and tendon/ligament as well as autologous chondrocyte implantation. Thus, administration of MSCs can be performed by administering the cells via a relatively pure population or in a construct generated using tissue engineering.

Administration of the Notch 2+ MSCs can promote bone formation following bone surgery, wherein the bone surgery is selected from the group consisting of facial reconstruction, maxillary or mandibular reconstruction, fracture repair, bone graft, prosthesis implant, joint replacement (e.g., hip and knee replacement).

Optionally, the Notch 2+ MSCs are differentiated (as described above) and delivered to an affected area of a subject. For example, osteogenic lineages can be delivered to a subject with a bone disease or defect.

Bone disorder or defect, as used herein, refers to any bone defect, disease or state which results in or is characterized by loss of health or integrity to bone and includes, but is not limited to, osteoporosis, osteopenia, faulty bone formation or resorption, Paget's disease, fractures and broken bones, bone metastasis, osteopetrosis, osteosclerosis and osteochondrosis. Bone defects and disorders include fractures and inherited or acquired disease states like osteogenesis imperfecta or osteoporosis. Bone diseases or defects that can be treated and/or prevented in accordance with methods described herein, include bone diseases characterized by a decreased bone mass relative to that of corresponding non-diseased bone (e.g., osteoporosis, osteopenia and Paget's disease). Cartilage defects include an articular cartilage defect or vertebral disc defect, which can be caused by trauma or diseases such as osteoarthritis or rheumatoid arthritis.

Treatment of a bone or cartilage defect or disorder or a symptom related to a bone or cartilage defect or disorder encompasses actively intervening after onset to slow down, ameliorate symptoms of, or reverse the disease or symptoms. Treating, as used herein, refers to a method that modulates bone or cartilage mass or integrity to more closely resemble that of corresponding non-affected bone (that is a corresponding bone of the same type, e.g., long and vertebral) or cartilage in a non-diseased or non-affected state. By way of example, following treatment post surgery, the bone or cartilage would resemble healthy, non-surgically affected bone.

The Notch 2+ MSCs can be administered in the form of a pharmaceutical composition. Such a composition comprises a therapeutically effective amount of the MSCs and a pharmaceutically acceptable carrier or excipient. Such a carrier includes but is not limited to, saline, buffered saline, dextrose, water, and combinations thereof. The formulation should suit the mode of administration. Optionally, the MSC composition is formulated for intravenous, intra-articular, or intervertebral administration. Compositions for intravenous administration are, for example, solutions in sterile isotonic aqueous buffer.

A composition including the Notch 2+ MSCs for use in the methods described herein can also be formulated as a sustained and/or timed release formulation. Such sustained and/or timed release formulations may be made by sustained release means, delivery devices or tissue-engineered constructs. The compositions can be used to provide slow or sustained release of one or more of the active ingredients using, for example, hydropropylmethyl cellulose, other polymer matrices, gels, permeable membranes, osmotic systems, multilayer coatings, microparticles, liposomes, microspheres or a combination thereof to provide the desired release profile in varying proportions. Various suitable sustained release formulations may be readily selected for use with the compositions described herein. Optionally, the compositions can be delivered by a controlled-release system. For example, the composition can be administered using intravenous infusion, an implantable osmotic pump, liposomes, or other modes of administration. A controlled release system can be placed in proximity of the target. For example, a micropump can deliver controlled doses directly into a joint or directly into bone or cartilage, thereby requiring only a fraction of the systemic dose (see e.g., Goodson, 1984, in Medical Applications of Controlled Release, vol. 2, pp. 115-138, which is incorporated by reference in its entirety at least for the material related to micropumps). In another example, the composition can be formulated with a hydrogel (see, e.g., U.S. Pat. Nos. 5,702,717; 6,117,949; 6,201,072, which are incorporated by reference in their entireties at least for the material related to hydrogels).

It may be desirable to administer the composition locally, i.e., to the area in need of treatment. Local administration can be achieved, for example, by local infusion during surgery, topical application, injection, or implant. An implant can be of a porous, non-porous, or gelatinous material, including membranes, such as sialastic membranes, or fibers and include tissue engineered constructs designed to replace tissues like bone or cartilage.

The Notch 2+ MSCs are used in an effective amount. In general, such amount ranges from at least 1×10⁴ MSC per kg of body weight to 3×10⁶ MSCs/kg of body weight. Optionally, the MSCs are administered at 1×10⁶ MSCs/kg of body weight. The MSCs are administered, for example, one to three times per day, and may be adjusted to meet optimal efficacy and pharmacological dosing. One of skill in the art can determine dosage amounts and frequency based on the route of administration; age, sex, health and weight of the recipient; nature and extent of symptoms; kind of concurrent treatment, frequency of treatment and the effect desired.

Also provided herein is a pack or kit comprising one or more containers filled with one or more of the ingredients (e.g., an activator of the Notch signaling pathway or Notch 2+ MSCs) described herein. Thus, for example, a kit described herein comprises a population of Notch 2+ MSCs. Also described is a kit with compositions for isolating Notch 2+ MSCs. Optionally, the kit further includes agents for culturing the Notch 2+ MSCs. Such kits optionally comprise solutions and buffers as needed or desired. Optionally associated with such pack(s) or kit(s) are instructions for use.

As used throughout, by a subject is meant an individual. Thus, the subject can include, for example, domesticated animals, such as cats and dogs, livestock (e.g., cattle, horses, pigs, sheep, and goats), laboratory animals (e.g., mice, rabbits, rats, and guinea pigs) mammals, non-human mammals, primates, non-human primates, rodents, birds, reptiles, amphibians, fish, and any other animal. The subject can be a mammal such as a primate or a human.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation may not be explicitly disclosed, each is specifically contemplated and described herein. For example, if a method is disclosed and discussed and a number of modifications that can be made to the method are discussed, each and every combination and permutation of the method, and the modifications that are possible, are specifically contemplated unless specifically indicated to the contrary. Likewise, any subset or combination of these is also specifically contemplated and disclosed. This concept applies to all aspects of this disclosure including, but not limited to, the MSCs themselves and steps in the methods of isolating, culturing and using the disclosed MSCs. Thus, if there are a variety of additional steps that can be performed it is understood that each of these additional steps can be performed with any specific method steps or combination of method steps of the disclosed methods, and that each such combination or subset of combinations is specifically contemplated and should be considered disclosed.

Throughout this application, various publications are referenced. The disclosures of these publications in their entireties are hereby incorporated by reference into this application.

A number of aspects have been described. Nevertheless, it will be understood that various modifications may be made. Furthermore, when one characteristic or step is described it can be combined with any other characteristic or step herein even if the combination is not explicitly stated. Accordingly, other aspects are within the scope of the claims.

### EXAMPLES

### Example 1

### RBPjk-Dependent Notch Signaling Maintains and Expands Mesenchymal Stem Cells (MSCs) During Skeletal Development

Materials and Methods

Mouse Strains.

All mouse strains including Rosa-NICD, Rbpjκ, and Prx1Cre are as previously described (Han et al., Int. Immunol. 14:637-45 (2002); Logan et al., Genesis 33:77-80 (2002); and Murtaugh et al., PNAS 100:14290-5 (2003)). Prx1Cre mice were obtained from the Jackson Laboratory (Bar Harbor, Me.).

Analyses of Mouse Embryos.

Embryonic tissues were harvested at E11.0-E12.5 in PBS, fixed in 10% neutral buffered formalin overnight at room temperature, then processed and embedded in paraffin prior to sectioning at 4 μm. Standard Alcian blue/orange g staining was performed in order to analyze tissue architecture and cartilage composition of the limb-buds. In situ hybridization was performed as described previously (Hilton et al., Development 132:4339-51 (2005); Hilton et al., Dev. Biol. 308:93-105 (2007); and Hilton et al., Nat. Med. 14:306-14 (2008)), using ³⁵S-labeled riboprobes. Unpublished riboprobes were generated from the following cDNA clones: Sox9 (4165469), Agc1 (5345931), Hes1 (10469606), Hey1 (9792713), Jag1 (10699187), Dll1 (10698888), and Dll4 (7492828). The cDNA clones are available from Open Biosystems (Huntsville, Ala.) or ATCC (Manassas, Va.). The Gfp probe was generated by cloning the enhanced Gfp coding sequence into the pGEM-T Easy vector. Notch1, Notch2, Notch3, Fgf8, and Fgf10 cDNAs and riboprobes are as described (Bellusci et al., Development 124:4867-78 (1997); Crossley and Martin, Development 121:439-51 (1995); and Mitsiadis et al., J. Cell Biol. 130:407-18 (1995)). For BrdU immunostaining analyses, pregnant females were injected with BrdU at 0.1 mg/g body weight 2 hours prior to harvest. BrdU detection was performed on paraffin sections using a kit from Zymed Laboratories (San Francisco, Calif.) as per manufacturer's instructions. Proliferation studies were confirmed using anti-Ki67 immunostaining (DAKO; Denmark) of mouse limb-bud paraffin sections according to manufacture's instructions. Analyses of apoptotic MSCs were performed using both anti-Cleaved Caspase-3 immunostaining (Cell Signaling; Danvers, Mass.) and TUNEL staining (Roche Cell Death In situ Kit; Roche; Basel, Switzerland) on limb-bud sections according to the manufacturers' instructions. Whole-mount skeletal staining of embryos was performed as previously described (Hilton et al., Development 132:4339-51 (2005); McLeod, Teratology 22:299-301 (1980)).

Limb-Bud MSC and C3H10T1/2 Cell Culture.

Limb-bud derived MSCs were isolated from E11.5 CD1 mouse embryos as previously described (Zhang et al., Bone 34:809-17 (2004)). For chondrogenic differentiation, MSCs were seeded in micromass (1×10⁵ cells in 10 Tl) in 12-well plates for 1.5 hours before adding standard media, media containing DAPT (1 μM), or media containing Hes1 shRNA lentivirus. Cells were cultured for a time-course of 6 hours, 3, 5, and 7 days prior to harvest for cartilage staining (1% Alcian blue/3% glacial acetic acid) or total RNA isolations. Limb-bud derived MSCs were also cultured in monolayer for 21 days and treated with either osteogenic (10 nM dexamethasone; 50 μM ascorbic acid; 10 mM β-glycerolphosphate) or adipogenic medium (Millipore; Billerica, Mass.) in the presence and absence of DAPT. Fixed MSCs were stained for osteoblastic differentiation using an alkaline phosphatase stain (nitro blue tetrazolium chloride/5-bromo-4-chloro-3-indolyhosphate P-toluidine salt) or adipogenic differentiation using an Oil Red-O staining solution (0.36%). Total RNA was isolated from monolayer cultures at day 21 for use in real-time RT-PCR analyses.

C3H10T1/2 cells were expanded and plated in monolayer for experiments as previously described (Denker et al., Differentiation 64:67-76 (1999); Haas and Tuan, Differentiation 64:77-89 (1999)). Monolayers were either transfected with 500 ng of CMV-Hes1 or CMV-control plasmid using the Lipofectamine 2000 reagent (Invitrogen; Carlsbad, Calif.) as suggested by the manufacturer's protocol, or infected with control virus or shRNA lentivirus against Hes1, Hey1, and HeyL (Sigma; St. Louis, Mo.). After 1 day of transfection/infection, cells were trypsonized and replated in micromass at a density of 1×10⁵ cells/10 Tl medium in each well of 12 well plates. Cells were harvested at days 5, 10, and 14 for Alcian blue staining and total RNA isolation.

Real-Time RT-PCR.

Embryonic limb-bud tissues or micromass cultures were frozen in liquid nitrogen and then homogenized in Trizol Reagent (Invitrogen; Carlsbad, Calif.) via rendering through a 25-gauge needle and syringe. Total cellular RNA was extracted following the manufacture's protocol. RNA was quantified using a NanoDrop spectrophotometer (NanoDrop; Wilmington, Del.) and equal concentrations of total RNA were pooled for synthesis of cDNA. Total RNA (1 μg) was reverse transcribed using the iScript™ cDNA synthesis kit (Bio-Rad; Hercules, Calif.) according to the manufacture's instructions. Reverse transcribed cDNA was analyzed by real-time RT-PCR with mouse-specific primers for: Sox9, Runx2, Col2a1, Agc1, Col1a1, Ap, Oc, Pparγ, Jagged1, Jagged2, Delta-like1, Delta-like3, Delta-like4, Notch1, Notch2, Notch3, Notch4, Hes1, Hes3, Hes5, Hes7, Hey1, Hey2, HeyL, and CyclinD1. Primers were designed using Applied Biosystems software (Applied Biosystems; Foster City, Calif.). Sequences are available upon request. DNA amplification was achieved using the SYBR® Green PCR Master Mix (Applied Biosystems; Foster City, Calif.) and the RotorGene real-time DNA amplification system (Corbett Research; Sydney, Australia). Gene expression was normalized to β-actin expression levels and then normalized to control samples.

Western Blot Analyses.

Total protein was isolated from either whole mouse limb-bud tissue or cultured limb-bud derived MSCs using Golden lysis buffer. The cultured limb-bud derived MSCs were plated at the density of 6×10⁶ cells in 10 cm dishes and cultured overnight in 10% FBS DMEM media both in the presence and absence of DAPT (1 um). Protein samples (˜100 μg) from each isolation were subsequently separated on 10% SDS-polyacrylamide and transferred to a PVDF membrane. NICD1 and NICD2 cleaved proteins were detected using the bTAN 20 (Notch1) and C651.6DdHN (Notch2) primary antibodies (0.4 ug/ml) and then further probed with appropriate secondary antibody (1:3000). Anti-β-actin antibody (Sigma; St. Louis, Mo.) was used as a control for equal protein loading. Immunoblots were detected using Supersignal west femto maximum sensitivity substrate (Pierce; Rockford, Ill.).

Results

Expression of Notch Pathway Components During MSC Differentiation In Vitro and In Vivo.

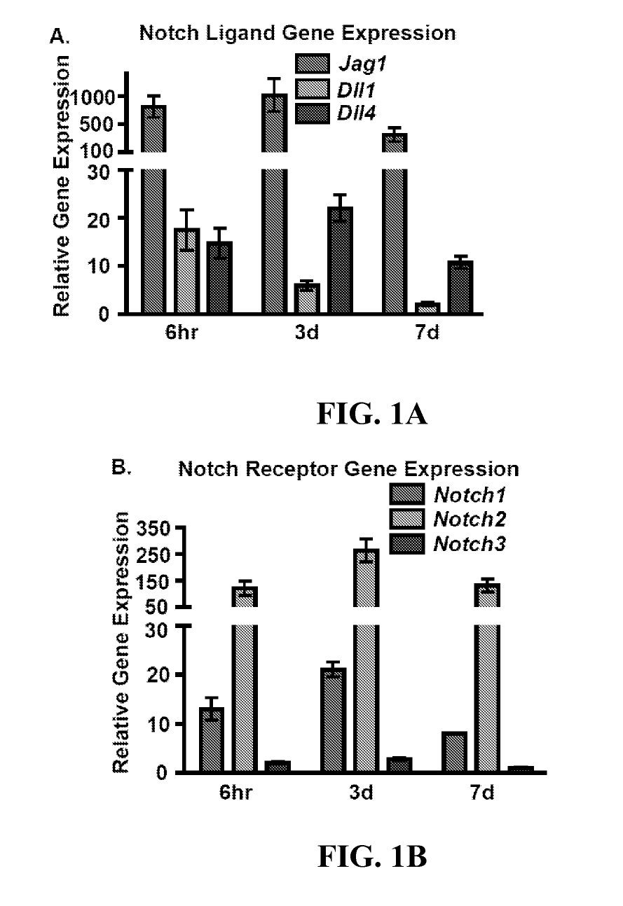

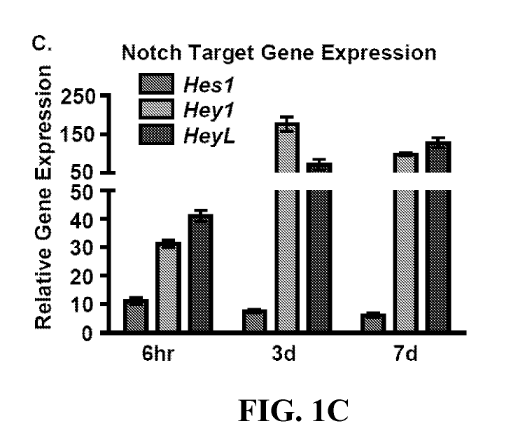

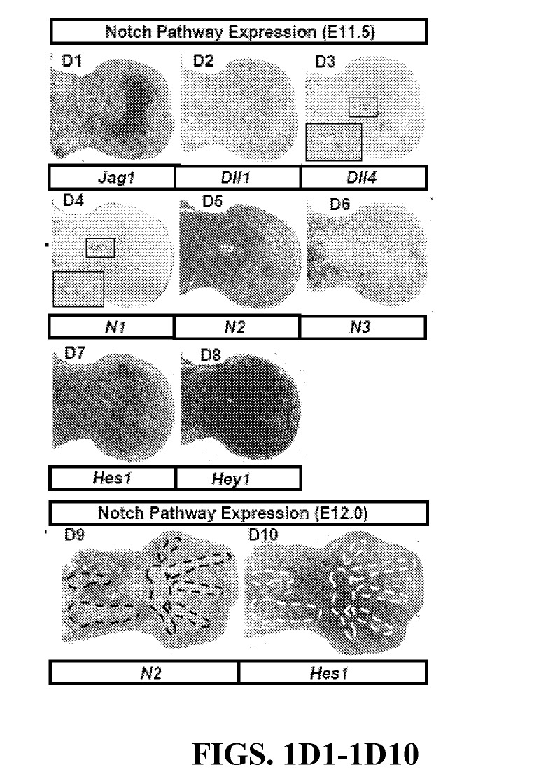

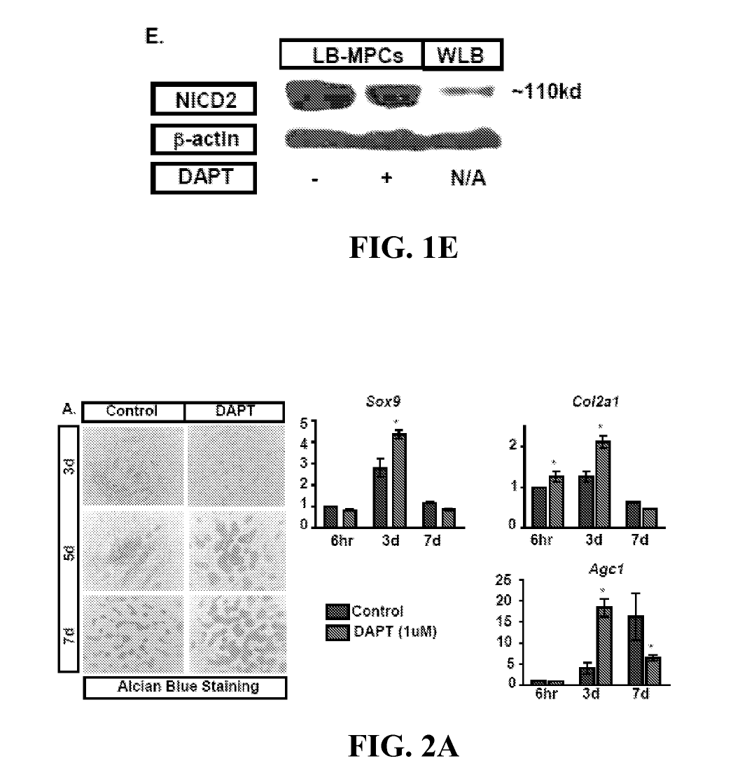

Real-time (RT) PCR was performed to identify the exact temporal expression of the five (5) murine Notch ligands (Jagged 1 (Jag1), Jagged 2 (Jag2), Delta-like 1 (Dll1), Delta-like 3 (Dll3), and Delta-like 4 (Dll4)), the four (4) Notch receptors (Notch 1 (N1), Notch 2 (N2), Notch 3 (N3), Notch 4 (N4)), and the six (6) canonical Notch target genes (Hes1, Hes5, Hes7, Hey1, Hey2, and HeyL) during limb-bud MSC differentiation and in vitro chondrogenesis. Limb-bud MSCs were isolated from E11.5 mouse embryos and cultured for 6 hours, 3 days, and 7 days in micromass. Of the five (5) possible Notch ligands, only Jag1, Dll1, and Dll4 were detected at significant levels, with Jag1 showing the highest level of expression at all time-points (FIG. 1A). Only three (3) of the four (4) Notch receptors (N1, N2, and N3) were detected during limb-bud MSC differentiation, with Notch2 displaying dramatically higher levels of expression at each time-point as compared to the other Notch receptors (FIG. 1B). To determine the downstream components of the Notch signaling pathway important during limb-bud MSC differentiation and chondrogenesis, the expression of RBPjκ-dependent Notch target genes was examined. Of the six (6) possible targets, only Hes1, Hey1, and HeyL were identified. Hey1 and HeyL were the most abundant Notch target genes showing similar levels of expression at each time-point that increased during MSC differentiation in vitro (FIG. 1C). While Hes1 displayed a lower level of expression as compared to Hey1 and/or HeyL, Hes1 expression was most pronounced in early limb-bud MSCs with declining expression levels during MSC differentiation, indicating a potential role in regulating the earliest stages of MSC commitment to the chondrocyte lineage (FIG. 1C).

In situ hybridization analyses was performed on E11.5 and E12.0 limb-bud sections to identify the exact in vivo spatial expression pattern for the Notch signaling molecules identified in the RT-PCR analyses. These data demonstrated that Notch ligands Jag1, Dll1, and Dll4 all had very different expression profiles. At E11.5, Jag1 was expressed moderately throughout much of the limb-bud mesenchyme but was highly expressed in a concentrated region of the distal, medial mesenchyme adjacent to the apical zone (FIG. 1D1). Of the other two Notch ligands, Dll1 was sporadically expressed throughout the limb-bud mesenchyme (FIG. 1D2), while Dll4 demonstrated a more concentrated expression pattern around vascular structures (FIG. 1D3, high magnification insert) at E11.5. Dll4 is a regulator of angiogenesis, which, along with Notch1, is a critical regulator of the vascular endothelium (Hellstrom et al., Nature 445:776-80 (2007); Shutter et al., Genes Dev. 14:1313-8 (2000)). The Notch receptor, Notch1, was also primarily expressed in regions of vascular tissues (FIG. 1D4, high magnification insert) and the early ectoderm at E11.5, with lower levels of expression observed throughout some of the limb-bud mesenchyme. Notch2 was expressed more ubiquitously throughout most of the limb-bud MSCs at the same stage (FIG. 1D5). Notch3 was expressed sporadically in the limb-bud mesenchyme, with higher concentrations in the proximal and peripheral MSCs. The Notch target genes, Hes1 and Hey1, each had expression patterns similar to that of Notch2 at E11.5 (FIGS. 1D5, 1D7, and 1D8), although a slight elevation of Hes1 expression could be observed in the distal, medial MSCs overlapping regions where Jag1 expression is concentrated (FIGS. 1D1 and 1D7). By E12.0-E12.5, most of the Notch pathway components are difficult to detect via in situ hybridization. Only Notch2 and Hes1 expression were maintained in limb-bud MSCs surrounding chondrogenic condensations, but showed significant down-regulation within the condensations themselves (FIGS. 1D9 and 1D10, black and white contours), while components like Hey1 maintained a more ubiquitous expression pattern.

To determine which Notch receptor is active in the limb-bud mesenchyme, total protein was isolated from cultured MSCs in the presence and absence of the Notch inhibitor, N-(3,5-difluorophenylacetyl-L-alanyl)]-S-phenylglycine t-ButylEster (DAPT, Calbiochem; San Diego, Calif.), or directly from wild-type E11.5 whole limb-bud tissue, and performed western blot analyses using Notch1 and Notch2 antibodies that can detect the cleaved or active (NICD) form of the receptor. Western blot analyses revealed that Notch2 was the prominent receptor activated in E11.5 limb-bud MSCs, and that DAPT treatment of cultured MSCs can reduce the abundance of the cleaved Notch2 (NICD2) (FIG. 1E). Notch1 (NICD1) was nearly undetectable at total protein concentrations up to 100 μg. Therefore, taken together these data suggest that Notch2 is the primary Notch receptor activated in MSCs, while other components of the Notch pathway (Jag1, Dll1, N3, Hes1, Hey1, and HeyL) may also be important mediators of MSC proliferation and differentiation during limb development.

Notch Signaling is a General Regulator of MSC Differentiation.

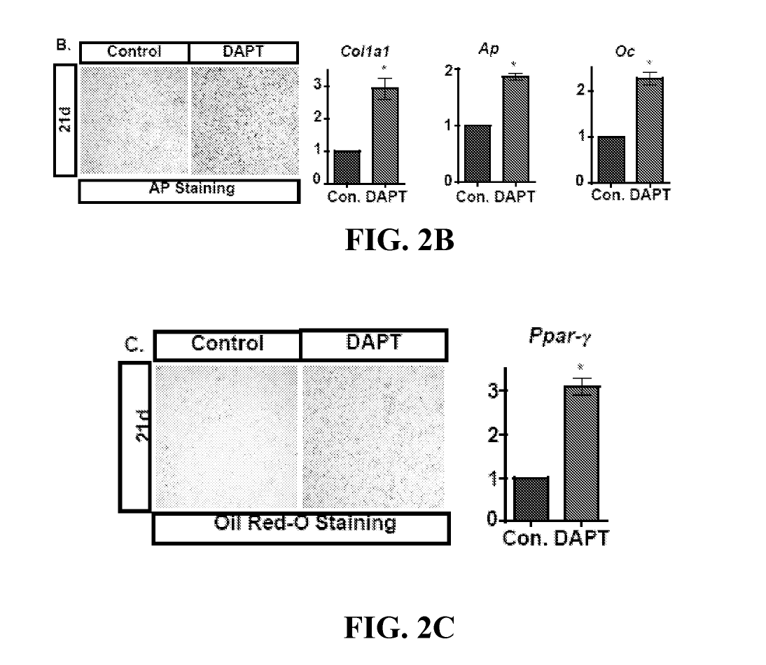

To determine the role of Notch signaling in MSCs, Notch loss-of-function assays were performed on E11.5 limb-bud derived MSC cultures using the Notch inhibitor, DAPT. Chondrogenesis was first examined in limb-bud micromass cultures by measuring cartilage nodule formation in the presence and absence of 1 μM DAPT. DAPT treatment significantly enhanced cartilage nodule formation (FIG. 2A), showing that Notch inhibition accelerates commitment of MSCs to the chondrocyte lineage, a finding that is consistent with a prior study (Fujimaki et al., J. Bone Miner. Metab. 24:191-8 (2006)). The effect of DAPT was also assessed on the expression of the chondrogenic markers Sox9, Col2a1, and Agc1 via real-time RT-PCR. Compared to untreated cultures, DAPT enhanced Sox9, Col2a1, and Agc1 expression (FIG. 2A) within the first 3-5 days of culture, although Agc1 expression was significantly reduced by day 7 indicating that Notch plays a later role in chondrocyte maturation or maintenance of the committed chondrocyte phenotype.

To determine whether Notch specifically regulates chondrogenesis or generally controls MSC differentiation, limb-bud MSC differentiation assays were performed in both osteogenic and adipogenic conditions. Limb-bud MSCs were plated in monolayer and cultured the cells for 21 days in osteogenic media in the absence and presence of DAPT (1 μM) (FIG. 2B). DAPT treatment enhanced normal osteoblastic differentiation of MSCs. Cultures displayed elevated alkaline phosphatase staining and real-time RT-PCR analyses demonstrated a significant increase in the expression of osteoblast marker genes: Col1a1, AP, and Oc (FIG. 2B). Finally, limb-bud MSCs were plated in monolayers and cultured the cells for 21 days in adipogenic media in the absence and presence of DAPT (1 μM) (FIG. 2C). DAPT treatment similarly enhanced normal adipogenic differentiation of MSCs. Cultures displayed elevated Oil Red-0 staining and real-time RT-PCR analyses demonstrated an increase in the expression of the adipocyte marker gene, Pparγ (FIG. 2C). These data demonstrate that inhibition of Notch signaling in vitro enhances limb-bud MSC differentiation toward the chondrocyte, osteoblast, and apipocyte lineages, showing a general role for Notch signaling in the maintenance of MSCs.

RBPjκ-Dependent Notch Signaling Suppresses MSC Differentiation During Chondrogenesis.

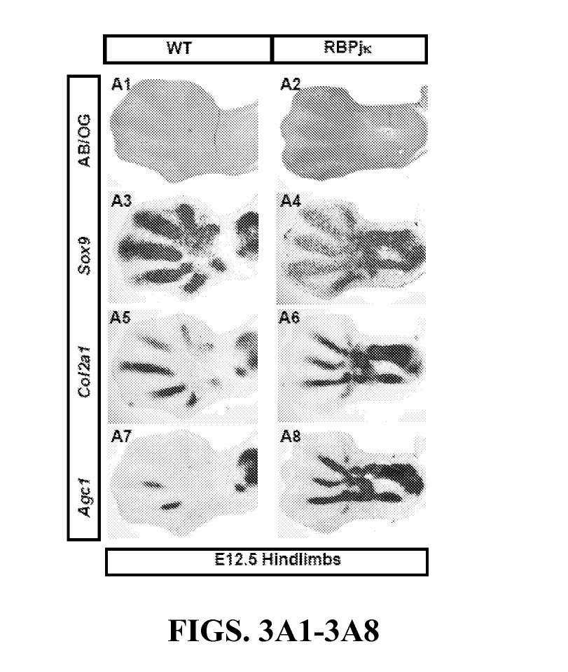

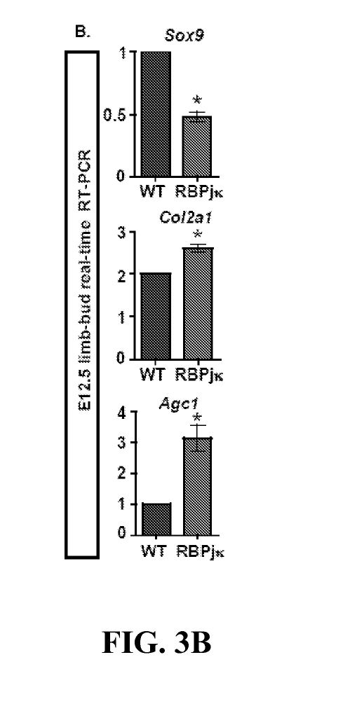

As a first step in assessing the requirement for Notch signaling during limb-bud MSC differentiation and chondrogenesis in vivo, embryonic mouse limb-buds were analyzed in which the canonical Notch effector, Rbpjκ, was selectively deleted in the early limb mesenchyme using the Prx1Cre transgene (Prx1Cre; Rbpjκ^{f/f} where “f” represents the floxed allele) (FIG. 3). The Prx1Cre mouse line was used in this study because it specifically targets MSCs of the lateral plate mesoderm that give rise to chondrocytes, osteoblasts, and connective tissue cells, but not myoblasts, blood lineage cells, or vascular endothelial cells within the developing limb. To assay for changes in the commitment of limb-bud MSCs to cells of the chondrocyte lineage, Alcian blue staining, in situ hybridization, and real-time RT-PCR were performed for Sox9, Col2a1, and Agc1. Prx1Cre; Rbpjκ^{f/f} mutant (RBPjκ) limb-buds at E12.5 exhibited an increase in Alcian blue staining of chondrogenic rudiments, as compared to controls that demonstrated nearly undetectable levels of Alcian blue staining (FIGS. 3A1 and 3A2). In situ hybridization analyses revealed an increase in both Col2a1 and Agc1 expression in RBPjκ mutant sections. All of the mutant Col2a1 positive cells also expressed Agc1 indicating that these cells are now fully committed chondrocytes (FIGS. 3A6 and 3A8). Wild-type sections at this stage demonstrated that only a central core of Col2a1 positive cells expressed Agc1, highlighting the normal progression of chondrocyte differentiation (FIGS. 3A5 and 3A7). Additionally, RBPjκ mutant sections displayed reduced levels of Sox9 expression suggesting that the mutant cells have progressed beyond the earliest stages of chondrogenesis. Real-time RT-PCR analyses performed on mRNA isolated from E12.5 whole limb-buds are consistent with the in situ hybridization results for each of the chondrogenic marker genes: Sox9, Col2a1, and Agc1 (FIG. 3B). Real-time RT-PCR performed on earlier limb-buds (E11.5) demonstrated elevated expression of all chondrogenic markers from RBPjκ mutant samples. These data suggest that RBPjκ-dependent Notch signaling normally maintains limb-bud MSCs, and that loss of RBPjκ results in accelerated chondrogenic differentiation for those cells determined to undergo the process of chondrogenesis.

Sustained Notch Activation Maintains and Expands MSCs in an Rbpjκ-Dependent Manner.

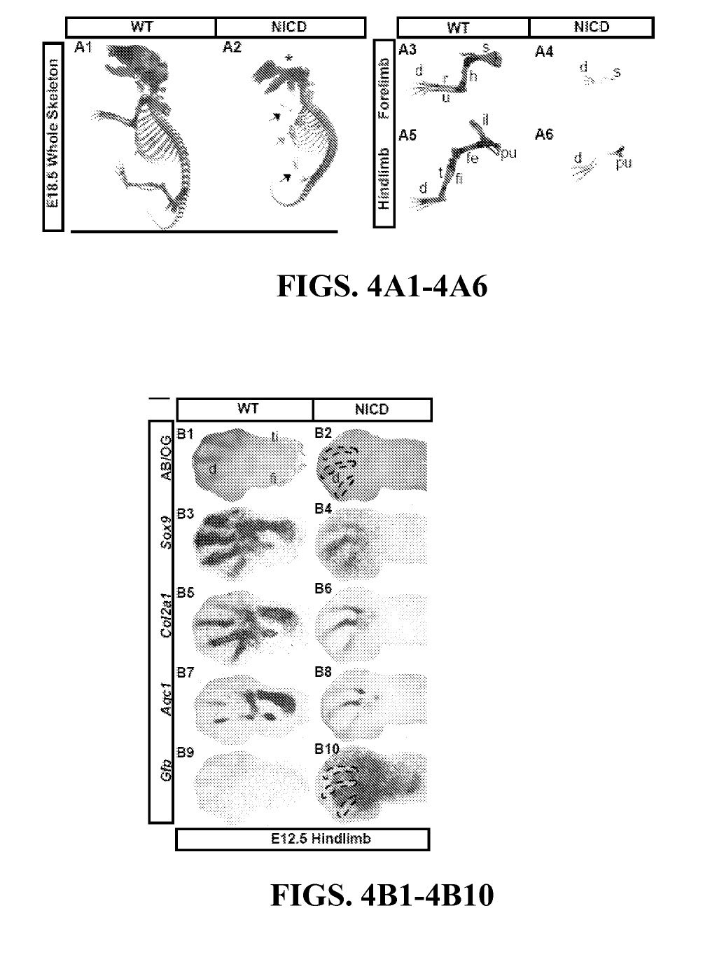

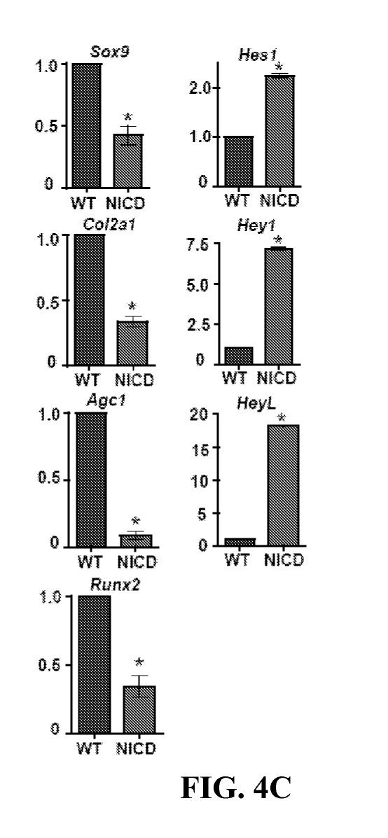

Notch gain-of-function experiments were performed to determine whether Notch activation in vivo could suppress or delay MSC differentiation and chondrogenesis in the developing limb. Gain-of-function experiments were performed using a mouse model system in which the intracellular domain of mouse Notch1 and GFP (NICD-IRES-GFP) were targeted to the Rosa26 Reporter locus containing upstream transcriptional stop sequences flanked by loxP sites (Rosa-NICD-IRES-GFP). It has been established that following Cre activation, the NICD and GFP expression is sustained specifically within Cre expressing cell populations (Murtaugh et al., PNAS 100:14920-5 (2003)). The Prx1Cre transgene was used to induce NICD expression and sustained Notch activity within the early limb-bud MSCs prior to chondrogenesis (Prx1Cre; Rosa-NICD^{f/+}), hereafter referred to as NICD mutants. Analyses of NICD mutant E18.5 skeletal preparations demonstrated a clear suppression of normal limb (black arrows), skull (asterisk), and sternum formation (gray arrow), all specific areas of Prx1Cre expression (FIGS. 4A1 and 4A2). Closer examination of the limbs revealed that only a few of the most proximal and distal cartilaginous rudiments developed in NICD mutants, although even these elements were hypoplastic with evidence of delayed cartilage development (FIG. 4A3-4A6). To determine if the limb phenotypes arose from the inhibition of MSC differentiation during chondrogenesis, E12.5 limb-buds were analyzed from NICD and WT control littermates. Sections from the NICD mutant limb-buds exhibited fewer condensations and thereby showed reduced Alcian blue staining as compared to controls (FIGS. 4B1 and 4B2). Mutants always displayed 3 digit condensations (apparent loss of 1^{st} and 5^{th} digits) and often did not develop more proximal condensations. When proximal condensations formed, they were always hypoplastic and were delayed in the chondrogenic differentiation process. To assess for disruptions in chondrogenesis and MSC differentiation, in situ hybridization was performed for Sox9, Col2a1, and Agc1. NICD mutant sections showed a near complete suppression of these marker genes, although the rudimentary digit condensations that did form seemed to express significant levels of each marker gene (FIG. 4B3-4B8). To investigate why these rudimentary condensations formed at all in the NICD mutants, in situ hybridization was performed for Gfp, which marks MSCs that actively express the NICD-IRES-GFP transcript and therefore have Notch activation. Each of the rudimentary condensations did not display evident Gfp expression while most other MSCs within the limb-bud showed robust Gfp expression, suggesting that the Prx1Cre transgene did not target this population of cells efficiently (FIGS. 4B9 and 4B10). RT-PCR analyses were performed on mRNA isolated from E12.5 whole limb-buds. These data are consistent with the in situ hybridization results for each of the chondrogenic marker genes, showing significant decreases in Sox9, Col2a1, and Agc1 expression (FIG. 4C). The expression of the early osteoblast differentiation regulator, Runx2, which like Sox9 showed significantly reduced levels of expression in the NICD mutants, was also performed (FIG. 4C). Analyses of the RBPjκ-dependent Notch target genes, Hes1, Hey1, and HeyL demonstrated increased levels of expression in NICD mutants as compared to WT littermate controls (FIG. 4C). These data suggest that Notch signaling suppresses MSC differentiation in a localized and possibly cell autonomous manner acting upstream of Sox9 and Runx2, potentially via RBPJκ-dependent signaling mechanisms.

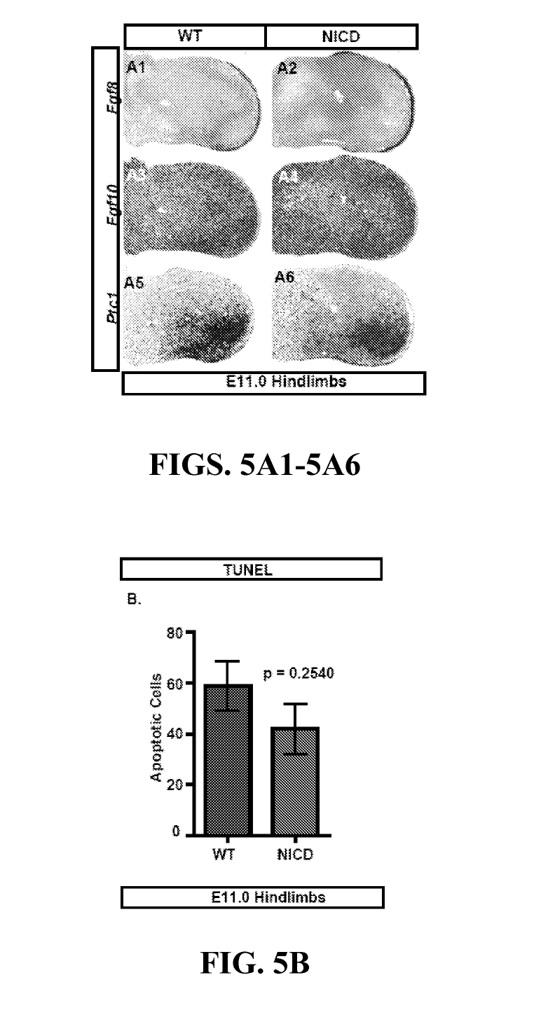

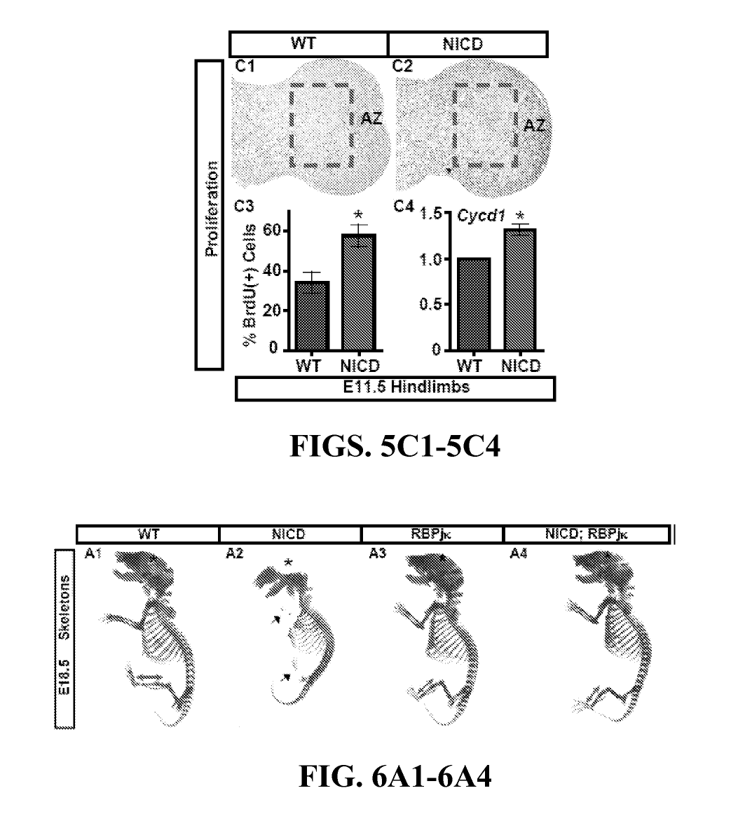

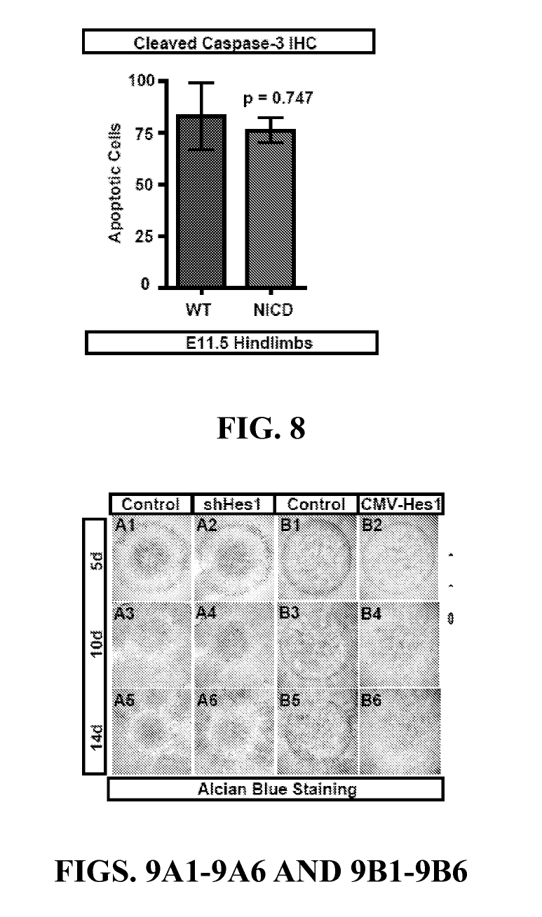

To exclude the possibility that sustained Notch activation impaired skeletal patterning and growth or massively induced MSC apoptosis, the expression of limb patterning regulators was analyzed and assessed alterations in proliferation and apoptosis. In situ hybridization studies were performed on E11.0 hindlimb sections for the FGF and Shh signaling molecules, Fgf8, Fgf10, and Ptc1, to determine whether critical regulators of limb development and patterning were significantly affected by NICD over-expression. While a slight thickening of the AER and an apparent increase in Fgf8 and Fgf10 expression was observed (FIG. 5A1-5A4), it was not thought that this can account for the cell autonomous suppression of MSC differentiation previously observed in these animals. Additionally, Patched1 (Ptc1) expression was unchanged between NICD mutant and WT sections (FIGS. 5A5 and 5A6) indicating uninterrupted Shh activity, which is critical for normal digit patterning and identity. TUNEL labeling and cleaved Caspase-3 IHC experiments were then performed to detect apoptotic MSCs on E11.0 hindlimb sections. NICD mutant sections showed no significant change in MSC apoptosis as compared to WT littermate controls (FIG. 5B and FIG. 8). No significant change in apoptosis at later time-points of MSC differentiation was detected. Finally, BrdU labeling experiments were performed on E11.5 hindlimb sections to determine whether sustained Notch activation has an adverse effect on MSC proliferation and limb growth. The data showed that NICD mutant sections displayed a significant increase in the percentage of BrdU labeled nuclei throughout the limb-bud, but was very evident in regions (dashed boxes) proximal to the highly proliferative apical zone (AZ) or progress zone (FIG. 5C1-5C3). To verify the BrdU data, RT-PCR was performed for the proliferation and cell cycle regulator, CyclinD1, using RNA derived from NICD mutant and control limb-buds at E11.5. NICD mutants exhibited a greater than 30% increase in CyclinD1 expression as compared to controls (FIG. 5C4). These data indicated that the limb phenotype in NICD mutants is likely caused by the cell autonomous suppression of MSC differentiation, and not due to perturbations in limb patterning, MSC apoptosis, or MSC proliferation. Furthermore, these data indicate that sustained Notch activation in limb-bud MSCs both maintains and expands this population of cells.

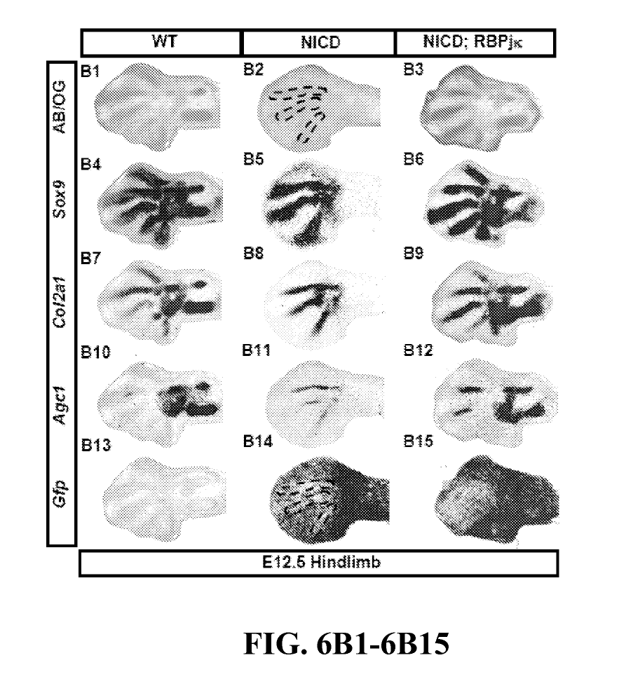

To determine whether Notch suppression of MSC differentiation and chondrogenesis was mediated solely via RBPjκ-dependent signaling mechanisms, Notch gain-of-function experiments were performed in the absence of the RBPjκ transcriptional effector. Mice carrying a Prx1Cre transgene, an activatable Rosa-NICD allele, and homozygous Rbpjκ floxed alleles (Prx1Cre; Rosa-NICD^{f/+}; Rbpjκ^{f/f}) were generated (NICD; RBPjκ). Analyses of alizarin red and Alcian blue stained skeletons at E18.5 demonstrated that in contrast to the NICD mutants which lacked normal limbs, specific skull bones, and sternum, the NICD; RBPjκ mutant animals failed to show a similar arrest in the development of these elements (FIGS. 6A1, 6A2, and 6A4). Upon closer examination, the NICD; RBPjκ mutant animals closely resembled the RBPjκ mutant skeletons, such that they had shorter skeletal elements (arrows highlight tibiae lengths) as compared to WT littermates (FIGS. 6 A1, 6A3, and 6A4). Detailed histological and molecular analyses of E12.5 hindlimb sections from WT, NICD, and NICD; RBPjκ mutant littermates further demonstrated that suppression of MSC differentiation via Notch activation requires RBPjκ. NICD mutants, which for this experiment had the genotype Prx1Cre; Rosa-NICD^{f/+} Rbpjκ^{f/+} displayed an identical phenotype to the previously described Prx1Cre; Rosa-NICD^{f/+} mutant mice (FIG. 6 NICD mutant compared to FIG. 4 NICD mutant). NICD mutants lacking a single Rbpjκ allele again demonstrated a near complete suppression of MSC differentiation resulting in limbs with only three distal digit condensations. E12.5 NICD limb-bud sections exhibited reduced Alcian blue staining and complete loss of chondrogenic marker gene expression (Sox9, Col2a1, and Agc1), except for within cells confined to the three distal digits (FIGS. 6 B2, 6B5, 6B8, and 6B11). When Gfp expression was assessed, once again the three digit condensations showed the near absence of Gfp expression and therefore a lack of sustained NICD activation (FIG. 6 B14). NICD mutants lacking both Rbpjκ alleles (NICD; RBPjκ) demonstrated a complete rescue of MSC differentiation and chondrogenesis. E12.5 NICD; RBPjκ mutant limb-bud sections showed the re-appearance of all chondrogenic elements with slightly expanded and more robust Alcian blue staining when compared to WT littermate controls (FIG. 6B1, 6B3). Additionally, in situ hybridization analyses of NICD, RBPjκ mutant sections demonstrated that the double mutants displayed accelerated and expanded Sox9, Col2, and Agc1 expression as compared to WT littermate controls, phenotypes strikingly similar to RBPjκ mutant littermates (FIGS. 6B4, 6B6, 6B7, 6B9, 6B10, and 6B12). To determine that the genetic rescue of MSC differentiation in NICD, RBPjκ mutants was not due to inefficient recombination and loss of NICD expression, in situ hybridization analyses were performed for Gfp expression on adjacent sections. NICD; RBPjκ mutant sections displayed robust levels of Gfp expression, and therefore NICD activation, throughout the limb-bud mesenchyme except for those regions previously identified in NICD mutant sections (FIG. 6B14, 6B15). Therefore, these data demonstrate for the first time that Notch suppression of MSC differentiation and chondrogenesis is solely mediated via RBPjκ-dependent signaling mechanisms.

The RBPjκ-Dependent Notch Target Gene, Hes1, is a Critical Regulator of MSC Differentiation During Chondrogenesis.

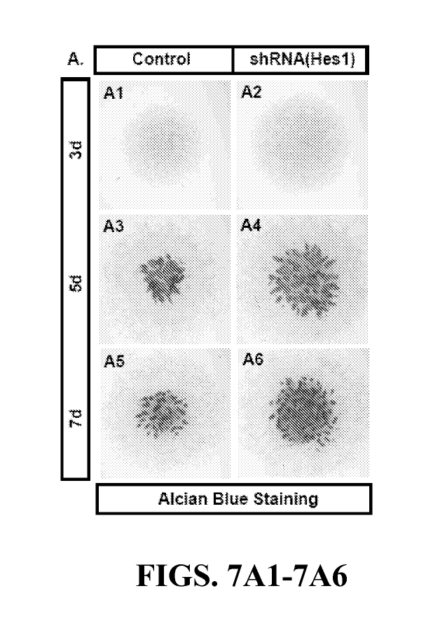

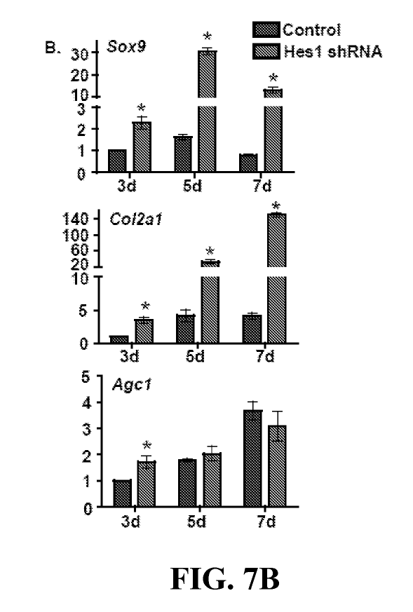

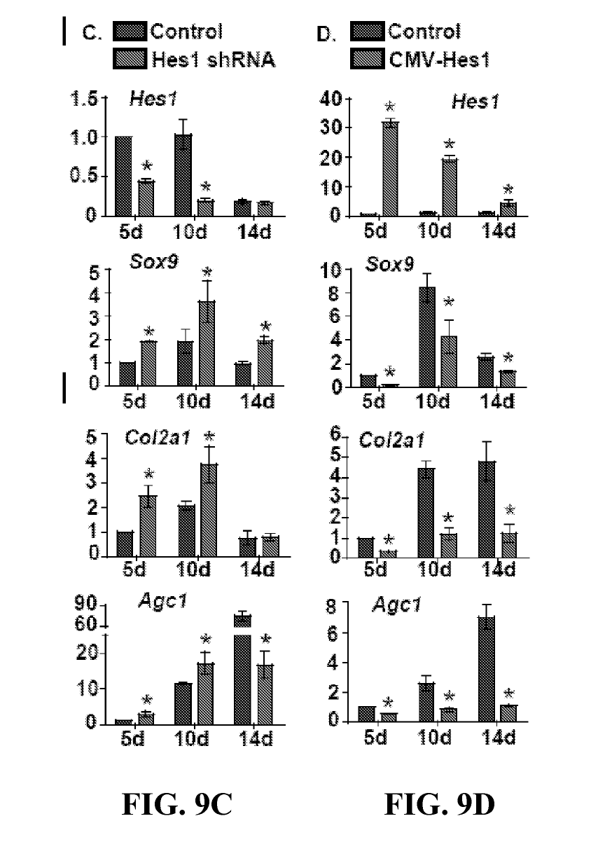

The data indicate that Notch regulation of chondrogenesis is mediated via RBPjκ-dependent Notch signaling mechanisms. Several RBPjκ-dependent Notch target genes of the Hes and Hey family mediate Notch control of stem/progenitor cell differentiation in several organ systems. Hes1, Hey1, and HeyL were the only classical Notch target genes significantly expressed in limb-bud MSCs and C3H10T1/2 mesenchymal cells cultured in high-density micromass (FIG. 1B). Therefore, loss-of-function experiments were performed by infecting the easily transducible C3H10T1/2 mesenchymal cells with Hes1, Hey1, and HeyL shRNA viruses while culturing in high-density micromass. Similar to limb-bud MSCs, the multi-potent mesenchymal cell line, C3H10T1/2, undergoes chondrogenesis when cultured in high-density micromass over a two-week culture period (Denker et al., Differentiation 64:67-76 (1999); and Haas and Tuan, Differentiation 64:77-89 (1999)). C3H10T1/2 cells transduced with Hes1 shRNA virus, and not Hey1 or HeyL shRNA virus, resulted in an acceleration or enhancement of chondrogenesis as assayed by Alcian blue staining and real-time RT-PCR for Sox9, Col2a1, and Agc1 (FIGS. 9A1-9A6 and 9C) similar to the other Notch loss-of-function studies. Hey1 and/or HeyL shRNA transduced cultures exhibited no significant change in Alcian blue staining, with inconsistent and relatively unchanged chondrogenic marker gene expression. Additionally, transient CMV-Hes1 over-expression gain-of-function experiments were performed in C3H10T1/2 micromass cultures, which demonstrated a significant suppression of chondrogenesis as assessed by Alcian blue staining (FIG. 9B1-9B6) and RT-PCR analyses were performed for each of the chondrogenic markers: Sox9, Col2a1, and Agc1 (FIG. 9D) similar to the other Notch gain-of-function studies. Since Hes1 appeared to be an important regulator of mesenchymal cell differentiation and chondrogenesis using the C3H10T1/2 cell model, analogous Hes1 shRNA loss-of-function studies were performed using limb-bud derived MSCs cultured in high-density micromass for 3, 5, and 7 days. Significant reductions in Hes1 expression resulted in accelerated chondrogenesis as observed by enhanced Alcian blue staning (FIG. 7A1-7A6) and elevated gene expression of the chondrogenic markers: Sox9, Col2a1, and Agc1 at nearly all time points in Hes1 shRNA cultures (FIG. 7B). At the later time-points, days 5 and 7, Agc1 expression was unchanged or mildly suppressed showing a role for Hes1 in promoting chondrocyte maturation or maintaining the committed chondrocyte phenotype. This was consistent with the experiments in which limb-bud derived MSCs cultured in high-density micromass were treated with the Notch inhibitor, DAPT (FIG. 2A). Collectively, these data showed that Hes1 is the primary RBPjκ-dependent Notch target gene of the Hes/Hey family expressed in MSCs and required for the Notch mediated suppression of MSC differentiation during chondrogenesis. Further, these data demonstrated that the RBPjκ-dependent Notch signaling pathway is critical for the maintenance and expansion of MSCs during skeletal development. Thus, manipulation of the Notch pathway provides a means to maintain, expand, and regulate the differentiation of MSCs ex vivo for the purpose skeletal repair and tissue engineering applications that utilize MSC populations.

### Example 2

### Notch Regulation of Human MSCs (hMSCs)

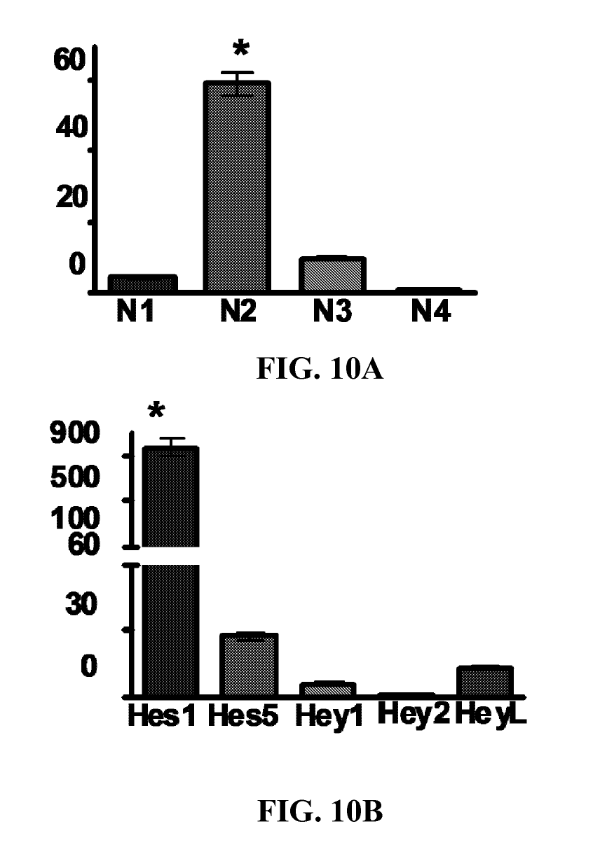

To explore how Notch signaling regulates hMSC maintenance and expansion, the expression profile for each Notch receptor and all known RBPjκ-dependent Notch target genes (Hes1, Hes5, Hes7, Hey1, Hey2, HeyL) from first passage, bone marrow derived hMSCs purchased from Lonza Inc. (Basel, Switzerland) (FIG. 10). All Notch receptors and most of the Hes/Hey target genes were expressed at variable levels. Notch2 (FIG. 10A) and Hes1 (FIG. 10B) were identified as the most highly expressed Notch components in hMSCs. This was consistent with the data from Example 1 analyzing Notch component expression and function in MSCs of the early developing mouse limb skeleton.

To demonstrate the ability to infect hMSCs with lentiviral constructs and induce Notch signaling in hMSCs via Jag1 coated plates, several control experiments were performed. hMSCs were first infected with the EF.v.CMV.GFP control lentivirus construct obtained from ATCC. This lentivirus expresses GFP allowing determination of infection efficiency after 24 hours and during multiple passages of the cells. The results demonstrated a greater than 85% infection efficiency within 24 hours, which is maintained during long-term cultures and continuous passages with no apparent change in hMSC growth or cell survival. A protocol for coating culture dishes with the recombinant Jag1 protein using 5 μg/ml, 10 μg/ml, and 15 μg/ml concentrations of Jag1 and 10 μg/ml concentration of IgG as controls was established. Immunostaining for the Jag1 protein on coated plates using an anti-Jag1 antibody and color reaction demonstrated that maximal and even coating of the plates was achieved at a concentration of 10 μg/ml recombinant Jag1. Higher concentrations did not appear to increase the yield of Jag1 bound to the culture dish. Alternatively, the 5 μg/ml concentration exhibited a Jag1 coating that appeared to be of significantly lower concentration, as well as, an uneven distribution of the protein around the periphery of the dish. IgG control plates also showed no color reaction as expected for a plate that did not contain the Jag1 recombinant protein. Next, to confirm that this Jag1 coating technology induced Notch signaling in hMSCs, hMSCs transfected with the RBPjP-dependent Notch luciferase reporter were cultured on 5 μg/ml, 10 μg/ml, and 15 μg/ml Jag1 and IgG coated plates. The data demonstrated that 10 μg/ml Jag1 protein induces maximal luciferase activity. It is also of note that the hMSCs appeared to grow normally on both the IgG and Jag1 coated plates with no obvious changes in cell size, shape, or cell survival.

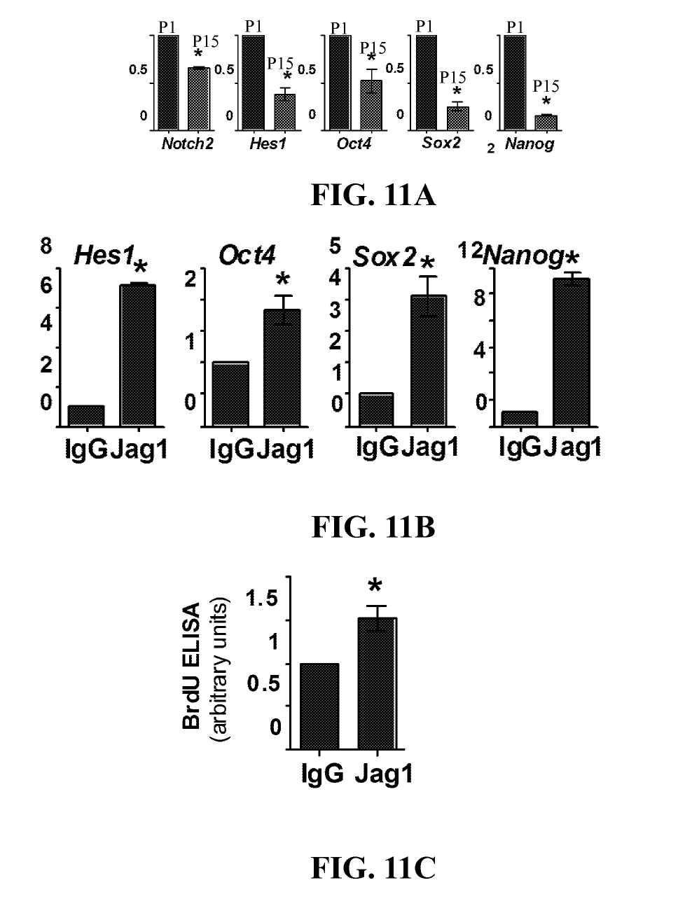

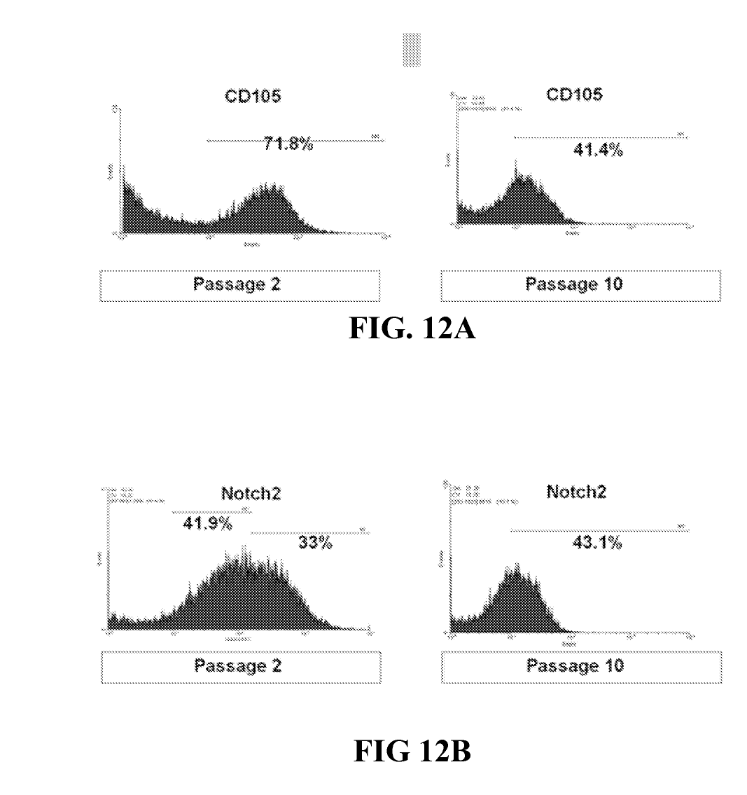

Since Notch signaling is a potent regulator of hMSC “stemness,” Notch molecules highly expressed in early passage hMSCs (Notch2 and Hes1) would change in their levels of expression as cells are passaged several generations, slowly losing their “stem-like” properties. The same rational would also apply to important regulators of “sternness” including Oct4, Sox2, and Nanog. Therefore, RT-PCR experiments were performed analyzing the gene expression of Notch2, Hes1, Oct4, Sox2, and Nanog from hMSCs that were passaged on normal culture plates in Mesenchymal Stem Cell Growth Medium (MSCGM™) (Lonza, Inc; Basel, Switzerland). The expression of these genes following passage 1 (P1) and passage 15 (P15) were compared. These data demonstrate that the Notch molecules (Notch2, Hes1) and the multipotent stem cell markers (Oct4, Sox2, Nanog) were significantly reduced in P15 hMSCs as compared to P1 (FIG. 11A). These data indicate a role for each of these factors in maintaining hMSC “stemness” during the ex vivo passaging of these cells. Flow cytometry data for hMSC cell surface marker CD105 and the Notch receptor, Notch 2 following passages 2 and 10 in standard hMSC culture conditions was also performed (FIGS. 12A and 12B).

To determine if Notch signaling can induce important regulators of stem cell maintenance, passage 3 hMSCs were cultured on Jag1 and IgG coated plates for 24 hours and isolated RNA for real-time RT-PCR analyses (FIG. 11B). The study showed that Jag1 coated plates (10 μg/ml) effectively induced RBPjP-dependent Notch signaling and enhanced Hes1 expression approximately 7-fold over controls. Additionally, Jag1 induced the expression of Oct4, Sox2, and Nanog, although Oct4 expression was only mildly enhanced compared to Sox2 and Nanog. Therefore, Jag1/Notch signaling regulated hMSC maintenance and expansion via this network of stem cell factors. Finally, the same culture system and passage 3 hMSCs were used to determine if Jag1 regulated the proliferation of hMSCs over a relatively short time interval. BrdU ELISA assays were performed for hMSCs cultured on Jag1 and IgG coated plates for 24 hours. The data demonstrated that Jag1 induced Notch signaling increases BrdU incorporation by more than 50% as compared to controls (FIG. 11C), showing that Notch signaling regulated both the maintenance and expansion of hMSCs ex vivo.

### Example 3

### Jagged1-Mediated Notch Activation in Notch-2 Selected hMSCs

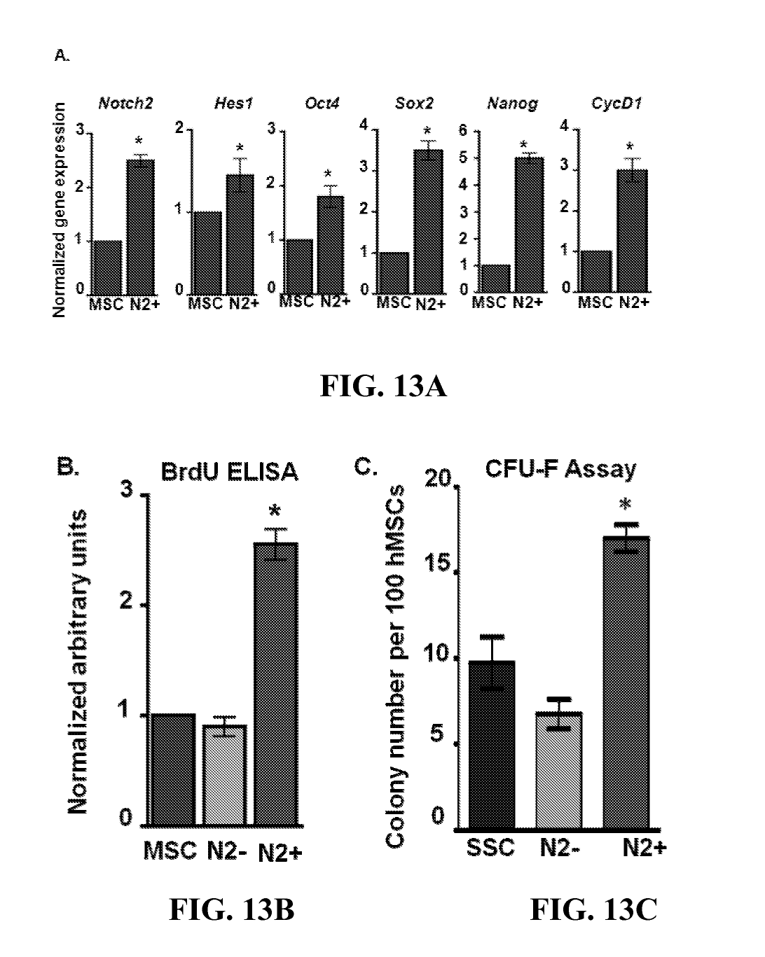

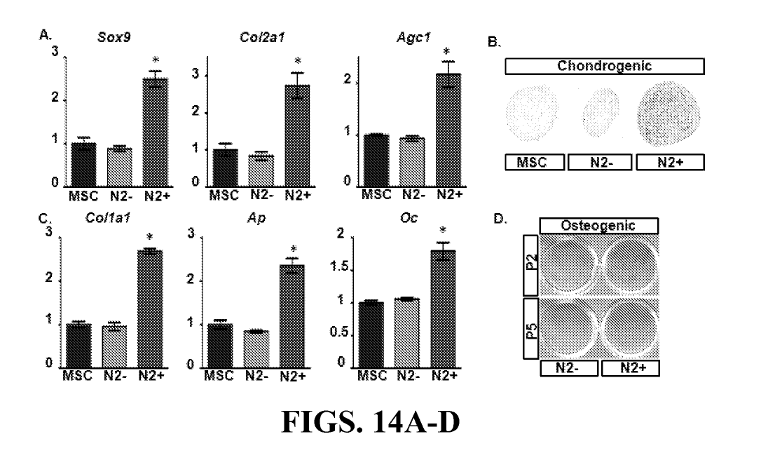

As shown in FIG. 13A, Jagged1-mediated Notch activation in Notch-2 selected hMSCs induced stem cell regulators, cell proliferation and stem cell expansion. More specifically, RT-PCR gene expression analysis for Notch signaling molecules (Notch 2 and Hes1), important stem cell regulatory molecules (Oct 4, Sox2 and Nanog) and a marker of cell proliferation (CycD1) in total hMSCs and Notch2-selected hMSCs culture on Jag1 coated plates showed increased gene expression in Notch2-selected hMSCs. FIG. 13B shows the results of a BrdU ELISA assay performed on total, Notch2-negative and Notch2-positive hMSCs cultured on Jag1 coated plates. Notch2-selected hMSCs showed increased proliferation as compared to total or Notch2-negative hMSCS. FIG. 13C shows the results of the CFU-F assay performed on total, Notch2-negative and Notch2-positive hMSCs cultured on Jag1 coated plates. Notch2-selected hMSCs showed increased stem cell expansion as compared to total or Notch2-negative hMSCS.

### Example 4

### Notch2-Selected hMSCs Display Enhanced Chondrogenic and Osteogenic Properties

As shown in FIGS. 14A and 14C respectively, Notch2-selected hMSCs displayed enhanced chondrogenic and osteogenic properties. Real-time RT-PCR gene expression analyses showed increases in chondrogenic (Sox9, Col2a1, and Agc1) (A) and osteogenic (Col1a1, Ap, and Oc) (C) marker genes in Notch2-positive hMSCS as compared to total and Notch2-negative hMSCs after being cultured in chondrogenic or osteogenic conditions for two to three weeks. Alcian Blue staining of total, Notch2-negative and positive hMSCs (Passage 2) following chondrogenic differentiation are shown in FIG. 14B. AP staining of Notch2-negative and positive hMSCs (Passage 2 and 5) following osteogenic differentiation are shown in FIG. 14D. hMSCs were initially cultured on Jag1 coated plates for two passages (3-4 days/passage).

These examples show that Notch2 and Hes1 are Notch signaling molecules expressed in human bone marrow derived MSCs (hMSCs). The expression of these Notch genes and important stem cell regulators decreased as hMSCs are passaged. Also shown is that Notch activation of hMSCs significantly induced not only the expression of Notch target genes, but also important stem cell regulatory molecules. Further, Notch2-selected hMSCs showed a superior induction of Notch pathway gene and stem cell regulatory molecule expression, proliferation, and stem cell expansion as compared to total or Notch2-negative hMSCs following Notch activation. Notch2-selected hMSCs also showed a superior ability to undergo chondrogenic and osteogenic differentiation as compared to total or Notch2-negative hMSCs after being removed from, for example, Jagged1-mediated hMSC maintenance and expansion.

### Example 5

### Effects of Ex Vivo Expanded Notch2 Positive Populations on Bone Defect Healing in a Femoral Allograft Mouse Model

In order to assess the effects of Notch2 positive populations in vivo, an adequate number of Notch2 positive mouse MSCs are generated using the novel MSC selection methods and Jagged1 induced MSC maintenance and expansion procedures described herein. Both Notch2-selected MSCs and total (traditionally selected) mouse MSCs are isolated from Rosa26LacZ mice so that the cells can be traced in vivo. Following maintenance and expansion, the MSCs are removed from the Jagged1 coated plates and the cells are seeded on devitalized allografts for transplantation into a femoral allograft mouse model of a critical segmented bone defect. Devitalized allograft without MSCs serve as a negative control group. On days 3, 7, 10, 14, 21, and 28 following transplantation, femurs are harvested from sets of mice (n=5-8) for use in X-ray, micro-CT, histology, immunohistochemistry (1HC), in situ hybridization (ISH), and lineage tracing (LacZ staining) analyses to assess the MSC incorporation, bone regeneration, and allograft osteointegration process. Biomechanical torsion testing is also performed at specific end-points to assess strength and integrity of the healing bones from each experimental and control group.

### Methods

Devitalization of Bone Allografts:

Ten week-old female mice of the 129 strain are obtained from Jackson Labs for donation of devitalized allografts. Briefly, mice are euthanized and a 4 mm mid-diaphyseal segment (about 20% of the femur length) is removed from each femur by osteotomy using a rotary Dremel and 2 parallel custom-fitted circular diamond blades with 4 mm spacing in between them. Allograft segments are flushed of the bone marrow using 25-gauge needles, the periosteum is manually stripped, and they are washed repeatedly in 70% ethanol for at least 4 hours. Allograft segments are inspected and the final removal of any remaining cells is performed if necessary. The allografts are stored in 100% ethanol at −80° C. for at least 30 days to complete the devitalization process.

Seeding of MSCs on Devitalized Allografts:

Following Jagged1-mediated MSC maintenance and expansion (as described above), Notch2-selected and total MSCs are seeded onto devitalized allografts. Briefly, the devitalized allografts are removed from the −80° C. freezer and allowed to equilibrate to room temperature. The grafts are placed in 96-well culture plates containing standard media for 30 min prior to the initial seeding of 5×10⁵ MSCs. MSCs are allowed to incubate for an additional 30 minutes at 37° C. in 5% CO₂ on the devitalized grafts. The grafts are rotated 180° and another 5×10⁵ MSCs are seeded onto the other side of the graft allowing for complete and even distribution of MSCs. The MSC seeded “revitalized” allografts are incubated at 37° C. in 5% CO₂ for about 1 hour to allow the cells to fully attach and integrate into the graft. The devitalized allografts that do not receive MSCs are placed in the same culture conditions prior to implantation. All devitalized and MSC revitalized bone allografts are then implanted into a 4 mm segmental defect created in the C57BL/6J recipient mice.

Surgical Reconstruction of the Mouse Femoral Defects:

Ten week-old female C57BL/6J mice are used in all experiments as allograft recipients. The mice are anesthetized via intraperitoneal injection with Ketamine (60 mg/kg body weight) and xylazine (4 mg/kg body weight). A 7-8 mm long lateral skin incision is made, and the mid-shaft of the femur is exposed by blunt dissection of muscles. A 4 mm mid-diaphyseal segment is removed from the femur by osteotomy as described above. The medullary canal is opened proximally and distally using a 22-gauge needle. The prepared devitalized allografts and MSC revitalized allografts are then inserted into the 4 mm defect and stabilized by a sterile Titanium pin which is placed through intramedullary marrow cavity. The intramedullary pin is bent both at the knee and at the hip to stabilize the pin. The incision is closed with interrupted silk sutures to allow for any initial imaging studies, following which the skin is closed with surgical staples. To control any acute pain induced by the bone grafting, buprenorphine (0.5 mg/kg) can be given post-operatively. Grafted samples are harvested at days 3, 7, 10, 14, 21 and 28 for evaluation of graft healing as well as MSC contribution to bone formation.

Micro-CT Bone Imaging Analyses:

Some of the reconstructed femurs from days 14, 21, and 28 (n=5) are imaged after careful dissection and removal of the intramedullary pin using a micro CT system (VivaCT 40, Scanco Medical). Briefly, the femurs are scanned using a protocol that utilizes high resolution (10.5 microns) x-ray energy settings of 55 kVp and 145 IA, an integration time of 200 milliseconds and a cone beam reconstruction algorithm. A region of about 8.00 mm (−800 slices) of the middiaphysis centered on the implanted allograft is scanned. Quantification of bone and graft volume and bone mineral density (BMD) is performed using the Scanco analysis software.

Biomechanical testing: After the micro CT imaging, specimens are moistened with saline and frozen at −20° C. until thawed for biomechanical testing. The ends of the femurs are cemented into 6.35 mm square aluminum tube holders using PMMA in a custom jig to ensure axial alignment and to maintain a gage length of 7-8 mm, allowing a length of at least 3 mm to be potted at each end. Specimens are bathed in PBS at room temperature for at least 2 hours after potting to allow for rehydration of the tissue and hardening of the PMMA. Specimens are mounted on an EnduraTec TestBench™ system (200 N.mm torque cell; Bose Corporation) and tested in torsion at a rate of 1°/sec until failure. The torque data is plotted against the rotational deformation (normalized by the gage length and expressed as rad/mm) to determine the Ultimate Torque (TUlt), yield torque, torsional rigidity (TR; which is computed from the slope of the linear region of the torque normalized rotational deformation curve), and torsional fracture energy (area under the torque-deformation curve). After testing to failure, all samples will be X-rayed to examine the mode of failure.

Histologic and Molecular Evaluation of Grafted Femurs:

The femoral samples that are to be used for histology and molecular analyses are fixed in neutral buffered formalin for 3 days, decalcified in 14% EDTA, pH 7.2, and processed in paraffin. Paraffin embedded samples (n=5) from days 3, 7, 10, 14, 21, 28 are sectioned at 5 μm. Several sections per block at defined depths within the healing femurs will be stained with OrangeG/alcian blue (H&E) to determine the contributions of cartilage, bone, and fibrotic tissue. Intervening unstained sections are used to perform in situ hybridization for specific markers of chondrocyte (Sox9, Col2a1, Agc1, Col10a1, and Mmp13) and osteoblast (Col1a1, Ap, Bsp, and Oc) differentiation using S-35 labeled riboprobes as previously described. Remodeling of the bone tissue will also be monitored using TRAP staining procedures. Histomorphometric analyses and quantification of areas of cellular staining and gene expression are performed using the OsteoMetrics system and OsteoMeasure software (see Tiyapatanaputi et al. A novel murine segmental femoral graft model. J Orthop Res 2004; 22-6:1254-60.)

Beta-Galactosidase Staining and MSC Lineage Tracing:

The femoral samples to be used for MSC lineage tracing analyses are fixed in 4% paraformaldehyde for 2 hours, decalcified in 14% EDTA, pH 7.2, processed through a 15% and 30% sucrose gradient, and frozen in O.C.T. embedding media. Frozen samples (n=3) from days 3, 14, and 28 will be sectioned at 8 μm. Sections from various depths of the healing femurs will be collected and stained for beta-galactosidase activity. Sections are analyzed as described below. Some of the frozen sections will be utilized for double labeling procedures to define the lineage of the LacZ stained cells. These sections will be first subjected to beta-galactosidase staining and then immediately used for in situ hybridization and/or immunohistochemistry with probes and antibodies specific for the chondrocyte (Col2a1, Col10a1) and osteoblast (Bsp, Oc) lineage. Staining, imaging, and image analysis of the dual labeled tissue sections is performed as previously described (see Hilton et al., Notch signaling maintains bone marrow mesenchymal progenitors by suppressing osteoblast differentiation. Nat Med 2008; 14-3:306-14).

Notch2-selected, maintained, and expanded mouse MSCs will exhibit a more robust effect on revitalized allograft incorporation and bone regeneration than revitalized allografts using traditionally selected MSCs or devitalized allografts alone as measured by X-ray, micro-CT, histology, IHC, ISH, LacZ staining, and biomechanical testing procedures. Furthermore, histological and molecular analyses will demonstrate that revitalized allografts with Notch2-selected MSCs exhibit an early enhancement in chondrogenic differentiation followed by an increase in osteoblast differentiation and accumulation of bone. The bone remodeling process will be similar in both of the revitalized allografts using Notch2-selected and total MSC populations as assessed by TRAP staining. Finally, beta-galactosidase staining and lineage tracing data from the revitalized allografts using Notch2-selected MSCs will show more chondrogenic and osteogenic differentiated cell lineages leading to enhanced bone formation directly from the donor cells as compared to revitalized allografts using total MSCs.

A number of embodiments have been described. Nevertheless, it will be understood that various modifications may be made. Accordingly, other embodiments are within the scope of the following claims.

1. A method of isolating from a subject a population of mesenchymal stem cells (MSCs) that maintain the capacity to expand through multiple passages, the method comprising:
(a) obtaining a biological sample comprising MSCs from the subject; and
(b) selecting for MSCs expressing a Notch 2 receptor from the biological sample to obtain a population of Notch 2+ MSCs.

2. The method of claim 1, wherein step (b) is carried out using a Notch 2 receptor antibody.

3. The method of claim 1, wherein step (b) is carried out using fluorescence activated cell sorting (FACS) or affinity chromatography.

4. The method of claim 2, wherein the Notch 2 receptor antibody is bound to a substrate.

5.-9. (canceled)

10. The method of claim 1, wherein the population of Notch 2+ MSCs express one or more additional markers associated with mesenchymal stem cells.

11. The method of claim 10, wherein the one or more additional markers are selected from the group consisting of CD73, CD90, CD105, CD106, CD156, CD44, CD29, CD166, Stro-1, FGF10, Prx1, Oct4, Sox2, and Nanog.

12. The method of claim 1, wherein the population of Notch 2+ MSCs do not express one or more markers associated with hematopoietic or endothelial cell lineage selected from the group consisting of CD11b, CD34, CD45, CD14, and CD31.

13. The method of claim 1, wherein the population of Notch 2+ MSCs express CD105 and CD156.

14. The method of claim 1, wherein the sample from the subject is selected from the group consisting of bone marrow, adipose tissue, synovium, periosteum, perichondrium, cartilage, dental tissue, placental tissue, liver tissue, muscle tissue, lung tissue, heart tissue, connective tissue, and spleen tissue.

15. The method of claim 14, wherein the sample is bone marrow.

16. A method of culturing the population of Notch 2+ MSCs derived by the method of claim 1 comprising culturing the MSCs in the presence of an activator of the Notch signaling pathway.

17. The method of claim 16, wherein the activator of the Notch signaling pathway is Jagged 1.

18. The method of claim 16, wherein the activator of the Notch signaling pathway is selected from the group consisting of delta-like 1, delta-like 3, delta-like 4, Jagged 2, Dlk1/Pref1, DNER, Contactin1 (F3), Contactin6 (NB3), CCN3/NOV, MAGP1, and MAGP2.

19. The method of claim 16, wherein the activator of the Notch signaling pathway is an intracellular domain of a Notch receptor.

20. The method of claim 19, wherein the Notch receptor is Notch 1, Notch 2, Notch 3, or Notch 4.

21. The method of claim 16, wherein the population of Notch2+ MSCs is expanded.

22. The method of claim 17, wherein the Jagged 1 is at least partially immobilized on a culture dish.

23. The method of claim 16, further comprising culturing the population of Notch 2+ MSCs in the presence of one or more differentiating agents.

24. The method of claim 23, wherein the one or more differentiating agents selectively induce differentiation into chondrogenic, osteogenic or adipogenic lineages.

25. A method of treating a subject with a disorder associated with a deficiency or defect in cells of mesenchymal lineage comprising administering a population of Notch 2+ MSCs to the subject.

26. The method of claim 25, wherein population of Notch2+ MSCs are derived from the same or a different subject.

27. The method of claim 25, wherein the population of Notch2+ MSCs is derived from the same subject.

28. The method of claim 25, wherein the subject has a bone or cartilage defect.

29. The method of claim 28, wherein the bone defect is a fracture or osteoporosis.

30. The method of claim 28, wherein the cartilage defect is an articular cartilage defect.

31. The method of claim 25, wherein the Notch 2+ MSCs are injected into the subject at or near the site of the bone or cartilage defect.

32. The method of claim 25, wherein the Notch 2+ MSCs are administered to the subject systemically.

33. A relatively pure population of MSCs expressing the Notch 2 receptor (Notch 2+ MSCs).

34. The Notch 2+ MSCs of claim 33, wherein the Notch 2+ MSCs maintain the capacity to expand through multiple passages.

35. The Notch 2+ MSCs of claim 33, wherein the Notch 2+ MSCs express CD105 and CD156.

36. The Notch 2+ MSCs of claim 33, wherein the Notch 2+ MSCs express one or more additional markers associated with mesenchymal stem cells selected from the group consisting of CD105, CD106, CD156, CD44, CD29, CD166, Stro-1, FGF10, Prx1, Oct4, Sox2, and Nanog.

37. The Notch 2+ MSCs of claim 33, wherein the Notch 2+ MSCs do not express one or more markers associated with hematopoietic or endothelial cell lineage selected from the group consisting of CD34, CD45, CD14, and CD31.

38. A relatively pure population of Notch 2+ MSCs made by the method of claim 1.

39. A method of treating a bone or cartilage defect in a subject comprising administering the Notch 2+ MSCs of claim 33 to the subject.

40. The method of claim 39, wherein the Notch 2+ MSCs are injected into the subject at or near the site of the bone or cartilage defect.

41. The method of claim 39, wherein the Notch 2+ MSCs are administered to the subject systemically.
